(19) United States
(12) Patent Application Publication
(10) Pub. No.: US 2024/0099332 A1
(43) Pub. Date: Mar. 28, 2024

(54) Methods and Compositions for Reducing Deleterious Enteric Atmospheric Gases in Livestock

(71) Applicant: Locus Solutions IPCo, LLC, Solon, OH (US)

(72) Inventors: Sean FARMER, Ft. Lauderdale, FL (US); Ken ALIBEK, Solon, OH (US); Karthik N. KARATHUR, Solon, OH (US); Keith HEIDECORN, Solon, OH (US)

(21) Appl. No.: 17/766,587

(22) PCT Filed: Feb. 10, 2021
(86) PCT No.: PCT/US2021/017399

§ 371 Date: Apr. 5, 2022

Related U.S. Application Data
(60) Provisional application No. 62/972,973, filed on Feb. 11, 2020.
(60) Provisional application No. 63/024,191, filed on May 13, 2020.
(60) Provisional application No. 63/038,985, filed on Jun. 15, 2020.
(60) Provisional application No. 63/126,711, filed on Dec. 17, 2020.

(51) Int. Cl. A23K 10/18; A23K 10/30; A23K 20/142; A23K 20/158; A23K 20/20; A23K 50/15; C02F 3/34; C05F 3/00; C12N 1/20

(52) U.S. Cl. CPC A23K 10/18; A23K 10/30; A23K 20/142; A23K 20/158; A23K 20/30; A23K 50/15; C02F 3/34; C05F 3/00; C12N 1/205; C02F 2103/20

(57) ABSTRACT

The subject invention provides compositions and methods for reducing deleterious atmospheric gas emissions produced in livestock animals' digestive systems and/or waste. In preferred embodiments, a composition comprising one or more beneficial microorganisms and/or one or more microbial growth by-products is contacted with the livestock animal's digestive system and/or waste in order to, for example, control methanogenic bacteria therein.

### CROSS-REFERENCE TO RELATED APPLICATIONS

This applications claims priority to U.S. Provisional Patent Application Nos. 62/972,973, filed Feb. 11, 2020; 63/024,191, filed May 13, 2020; 63/038,985, filed Jun. 15, 2020; and 63/126,711, filed Dec. 17, 2020, each of which is incorporated herein by reference in its entirety.

### BACKGROUND OF THE INVENTION

Gases that trap heat in the atmosphere are called “greenhouse gases,” or “GHG,” and include carbon dioxide, methane, nitrous oxide and fluorinated gases (EPA report 2016 at 6).

Carbon dioxide (CO₂) enters the atmosphere through burning fossil fuels (coal, natural gas, and oil), solid waste, trees and wood products, and also as a result of certain chemical reactions, e.g., the manufacture of cement. Carbon dioxide is removed from the atmosphere by, for example, absorption by plants as part of the biological carbon cycle.

Nitrous oxide (N₂O) is emitted during industrial activities and during combustion of fossil fuels and solid waste. In agriculture, over-application of nitrogen-containing fertilizers and poor soil management practices can also lead to increased emissions of nitrous oxide and other nitrogen-based gases.

Fluorinated gases including, e.g., hydrofluorocarbons, perfluorocarbons, sulfur hexafluoride, and nitrogen trifluoride are synthetic, powerful greenhouse gases that are emitted from a variety of industrial processes.

Methane (CH4) is emitted during the production and transport of coal, natural gas, and oil. Furthermore, other agricultural practices, and the decay of organic waste in lagoons and municipal solid waste landfills can produce methane emissions. Notably, however, methane emissions also result from production of livestock animals, many of whose digestive systems comprise methanogenic microorganisms (Overview of Greenhouse Gases 2016).

Based on recent measurements from monitoring stations around the world and measurement of older air from air bubbles trapped in layers of ice from Antarctica and Greenland, global atmospheric concentrations of GHGs have risen significantly over the last few hundred years (EPA report 2016 at, e.g., 6, 15).

Especially since the Industrial Revolution began in the 1700s, human activity has contributed to the amount of GHGs in the atmosphere by burning fossil fuels, cutting down forests, and conducting other activities. Many GHGs emitted into the atmosphere remain there for long periods of time ranging from a decade to many millennia. Over time these gases are removed from the atmosphere by chemical reactions or by emissions sinks, such as the oceans and vegetation that absorb GHGs from the atmosphere.

World leaders have attempted to curb the increase of GHG emissions through treaties and other inter-state agreements. One such attempt is through the use of carbon credit systems. A carbon credit is a generic term for a tradable certificate or permit representing the right to emit one ton of carbon dioxide, or an equivalent GHG. In a typical carbon credit system, a governing body sets quotas on the amount of GHG emissions an operator can produce. Exceeding these quotas requires the operator to purchase extra allowances from other operators who have not used all of their carbon credits.

One goal of carbon credit systems is to encourage companies to invest in more green technology, machinery and practices in order to benefit from the trade of these credits. Under the Kyoto Protocol of the United Nations Framework Convention on Climate Change (UNFCCC), a large number of countries have agreed to be bound internationally by policies for GHG reduction, including through trade of emissions credits. While the United States is not bound by the Kyoto Protocol, and while there is no central national emissions trading system in the U.S., some states, such as California and a group of northeastern states, have begun to adopt such trading schemes.

Another attempt to reduce atmospheric GHGs, in particular, methane emissions, has involved the use of feed additives or supplements in livestock production. Ruminant livestock, such as, for example, cattle, sheep, buffalo, goats, deer and camels, are unique because of their four stomach compartments: the reticulum, rumen, omasum and abomasum. The rumen, in particular, is a large, hollow organ where microbial fermentation of ingested substances, such as fibrous plant material, occurs. This organ can hold 40-60 gallons of material, with an estimated microbial concentration of 150 billion microbes per teaspoon of rumen contents.

The rumen functions as an anaerobic fermentation vessel for certain bacteria that produce gaseous fermentation by-products, including oxygen, nitrogen, H₂ and carbon dioxide. See FIG. 1. Methanogenesis is a natural process contributing to the efficiency of the digestive system, reducing the partial pressure of H₂ and allowing the normal functioning of microbial enzymes. The process is regulated by methanogens, the most common of which is Methanobrevibacter. Methanogens form a biofilm on surfaces where hydrogen-producing bacteria and protozoa actively produce H₂ required for reducing carbon dioxide to methane.

As an example, cattle, raised for both beef and milk, as well as for inedible outputs like manure and draft power, are responsible for the greatest amounts of emissions from livestock, representing about 65% of the livestock sector's emissions. Approximately 130 to over 250 gallons of ruminal gas produced by fermentation can be belched from one cow each day. This is important for the health of the cow, as it prevents bloating; however, the negative result is the emission of GHG such as carbon dioxide and methane into the atmosphere.

Other animals, including non-ruminant animals, also contribute to enteric GHG production. For example, swine, rodents, monkeys, horses, mules, asses, rhinoceros, hippopotamuses, bears, poultry and certain other birds also contain methanogenic bacteria in their digestive systems. Certain monogastric animals also produce N₂O and CO₂ emissions.

In addition to gut fermentation, livestock manure can also be a source of GHG emissions. Manure contains two components that can lead to GHG emissions during storage and processing: organic matter that can be converted into methane emissions, and nitrogen that leads indirectly to nitrous oxide emissions. Methane is released when methanogenic bacteria decompose the organic material in the manure as it is being held in lagoons, tailing ponds or holding tanks. Additionally, nitrogen in the form of ammonia (NH₃) is released from manure and urine during storage and processing. Ammonia can later be transformed into nitrous oxide. (Gerber et al. 2013).

Currently, approaches for reducing livestock methane emissions include defaunation of the digestive system and even vaccination against methanogens. The downsides to these strategies, however, are that they may reduce the number of beneficial gut microbes, and the methods may be short-lived due to microbial adaptation. Additionally, energy providers have attempted to harvest methane from manure lagoons and collection ponds as a form of biogas fuel; however, the methods are inefficient and do not capture significant amounts of methane relative to the total amount of methane produced by livestock production.

Other strategies have involved dietary modification, particularly for livestock grazing pasture, in order to manipulate gut fermentation by, for example, directly inhibiting methanogens and protozoa, or by redirecting hydrogen ions away from the methanogens to reduce methanogenesis. Such dietary modifications include, for example, the addition of probiotics, acetogens, bacteriocins, ionophores (e.g., monensin and lasalocid), organic acids and/or plant extracts (e.g., tannins and/or seaweed), to feed. (Ishler 2016). Most anti-methanogenic compounds are costly, short-lived, show inconsistent results, require high concentrations, do not contain H₂ acceptors, do not affect methanogens in the form of biofilms, and comprise compounds that are easily destroyed and/or excreted.

Specific feed additive products include, for example, Mootral, Bovaer®, and FutureFeed, each of which has its own limitations. Mootral is a feed supplement comprising a proprietary combination of active compounds from garlic, and flavonoids derived from citrus. The product works by direct inhibition/killing of methanogenic bacteria. The amount of actual methane reduction, about 30%, is low however, compared to the cost per cow per year. Additionally, the product can increase the urea content of milk, indicating an increase in nitrous oxide production.

Bovaer® is a feed additive comprising 3-nitrooxy propanol, which inhibits methyl coenzyme M reductase, an enzyme that catalyzes the final step in methanogenesis. The amount of actual methane reduction achieved is also relatively low, at about 30%, and relatively short-term in duration. Additionally, due to a lack of H₂ acceptors, the product can cause a fishy smell in produced milk because of an increase in trimethylamine production.

FutureFeed is a feed supplement that utilizes a specific type of seaweed that can reduce enteric methane emissions by up to 98%. Nonetheless, the product is costly at $200 per kg and is slow to have a consistent impact on enteric gas production.

The livestock industry is important for the production of, for example, meats and dairy products; however, growing concerns over climate change and a need for reducing GHG emissions calls for improved approaches for producing livestock with reduced GHG emissions.

### BRIEF SUMMARY OF THE INVENTION

The subject invention provides compositions and methods for reducing atmospheric greenhouse gas emissions from livestock animals. More specifically, the subject invention provides compositions that, when contacted with the digestive system and/or waste of a livestock animal, lead to a reduction in greenhouse gas (GHG) emissions that would have otherwise been produced by the animal's digestive processes and/or waste. Advantageously, the compositions and methods can also enhance the overall health of a livestock animal.

In specific embodiments, the subject invention provides a digestive health composition for reducing enteric methane, carbon dioxide, and/or other deleterious atmospheric gases and/or precursors thereof produced in a livestock animal's digestive system and/or waste, wherein the composition comprises one or more beneficial microorganisms and/or one or more microbial growth by-products. In preferred embodiments, the beneficial microorganisms are non-pathogenic fungi, yeasts and/or bacteria capable of producing one or more of the following: surface active agents, such as lipopeptides and/or glycolipids; bioactive compounds with antimicrobial and immune-modulating effects; polyketides; acids; peptides; anti-inflammatory compounds; enzymes, such as proteases, amylases, and/or lipases; and sources of amino acids, vitamins, and other nutrients.

Advantageously, in preferred embodiments, the subject compositions can help reduce deleterious atmospheric gas emissions resulting from livestock production by controlling and/or inhibiting methanogenic microbes, and/or symbionts thereof, present in the animal's digestive system and/or waste.

In one embodiment the composition disrupts methanogen biofilms. In one embodiment, the composition directly inhibits methanogens and/or the biological pathway involved in methanogenesis.

Advantageously, in preferred embodiments, the subject compositions can also decrease the amount of excess H₂ that can be produced when methanogenesis is inhibited, by, for example, introducing H₂ acceptors.

In certain embodiments, the composition can be formulated for enteral and/or parenteral administration to the livestock animal's digestive system. For example, in certain embodiments, the composition can be formulated as dry feed pellets, powders and/or granules to supplement grains and/or forage (e.g., pasture plants, hay, silage and/or crop residue).

In certain preferred embodiments, the composition comprises one or more microorganisms, and/or growth by products thereof, wherein the microorganisms are bacteria, fungi and/or yeasts. The microorganisms can be, for example, Bacillus spp. bacteria; myxobacteria; Pleurotus spp. fungi; Lentinula spp. fungi; Trichoderma spp. fungi; Saccharomyces spp. yeasts; Debaryomyces hansenii; Starmerella bombicola; Wickerhamomyces anomalus; Meyerozyma guilliermondii; Pichia occidentalis; Monascus purpureus; and/or Acremonium chrysogenum.

The bacteria, when present, can be used in spore form, as vegetative cells, and/or as a mixture thereof.

The fungi can be in the form of live or inactive cells, mycelia, spores and/or fruiting bodies. The fruiting bodies, if present, can be, for example, chopped and/or blended into granules and/or a powder form.

The yeast(s) can be in the form of live or inactive cells or spores, as well as in the form of dried and/or dormant cells (e.g., a yeast hydrolysate).

In a preferred embodiment, the composition comprises a strain of B. amyloliquefaciens. In a specific preferred embodiment, the strain of B. amyloliquefaciens is B. amyloliquefaciens NRRL B-67928 (“B. amy”). B. amy is particularly advantageous over traditional probiotic microorganisms due to its ability to produce spores that remain viable in the digestive tract and, in some embodiments, after being excreted in the animal's waste. Additionally, B. amy produces a unique mixture of metabolites that provide a broad-spectrum of digestive and environmental benefits when administered to a livestock animal and/or its waste.

In one exemplary embodiment, the composition comprises B. amy. In one exemplary embodiment, the composition comprises B. subtilis “B4.” In one exemplary embodiment, the composition comprises Pleurotus ostreatus. In one exemplary embodiment, the composition comprises Saccharomyces boulardii. In one exemplary embodiment, the composition comprises Debaryomyces hansenii.

In certain exemplary embodiments, the composition can comprise any combination of B. amy, P. ostreatus, S. boulardii and/or D. hansenii.

In one embodiment, the digestive health composition comprises a microbial growth by-product. The microbial growth by-product can be produced by the microorganisms of the composition, and/or they can be produced separately and added to the composition.

In one embodiment, the growth by-product has been purified from a cultivation medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites and/or other biochemicals produced as a result of cell growth, including, for example, biosurfactants, enzymes, polyketides, acids, alcohols, solvents, proteins, and/or peptides.

In certain embodiments, the composition comprises a germination enhancer for enhancing germination of spore-form microorganisms used in the microbe-based composition. In specific embodiments, the germination enhancers are amino acids, such as, for example, L-alanine and/or L-leucine. In one embodiment, the germination enhancer is manganese.

In one embodiment, the composition comprises one or more fatty acids. In certain preferred embodiments, the fatty acid is a saturated long-chain fatty acid, having a carbon backbone of 14-20 carbons, such as, for example, myristic acid, palmitic acid or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In some embodiments, the composition can comprise additional components known to reduce methane in the livestock animal's digestive system, such as, for example, seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producer); Quillaja saponaria extract (triterpenoid saponin-producing plant species); organosulfurs (e.g., garlic extract); flavonoids (e.g., quercetin, rutin, kaempferoi, naringin, and anthocyanidins; biofiavonoids from green citrus fruits, rose hips and black currants); carboxylic acid; and/or terpenes (e.g., d-limonene, pinene and citrus extracts).

In one exemplary embodiment, the composition comprises a microorganism such as, e.g., B. amy, and 3-nitrooxypropanol (3NOP), an organic compound having the formula HOCH₂CH₂CH₂ONO₂. 3NOP is effective for suppressing one or more enzymes involved in methanogenesis, e.g., methyl coenzyme M reductase.

In one embodiment, the subject composition can comprise one or more additional substances and/or nutrients to supplement the livestock animal's nutritional needs and promote health and/or well-being in the livestock animal, such as, for example, sources of amino acids (including essential amino acids), peptides, proteins, vitamins, microelements, fats, fatty acids, lipids, carbohydrates, sterols, enzymes, minerals such as calcium, magnesium, phosphorus, potassium, sodium, chlorine, sulfur, chromium, cobalt, copper, iodine, iron, manganese, molybdenum, nickel, selenium, and zinc, and/or other bioactive compounds with anti-inflammatory, antimicrobial and/or immune-modulating effects in animals. In some embodiments, the microorganisms of the composition produce and/or provide these substances.

In preferred embodiments, the subject invention provides a method for reducing deleterious atmospheric gas emissions by reducing methane, carbon dioxide and/or other deleterious atmospheric gases, and/or precursors thereof (e.g., nitrogen and/or ammonia, which are precursors of nitrous oxide), produced in the digestive systems and/or waste of livestock animals.

In certain specific embodiments, the methods comprise contacting a digestive health composition according to the subject invention with the digestive system of a livestock animal. The composition can be administered enterally and/or parenterally to the livestock animal's digestive system. For example, the composition can be administered to the livestock animal orally, via the animal's feed and/or drinking water; via endoscopy; via direct injection into one or more parts of the digestive system; via suppository; via fecal transplant; and/or via enema.

Advantageously, in preferred embodiments, the methods result in a direct inhibition of methanogenic bacteria and/or symbionts thereof, disruption of methanogenic biofilms, and/or disruption of the biological pathway involved in methanogenesis in the livestock animal's digestion system, for example, the rumen, stomach and/or intestines.

In certain embodiments, the methods can also counteract Hr-acceptor depletion that results from reduced methanogenesis. Accordingly, potential negative effects of excessive H₂ on livestock products can be prevented and/or reduced. For example, excess H₂ in the digestive tract of mammals can produce a fishy smell in milk due to the overproduction of trimethylamine.

In some embodiments, the methods result in increased conversion of nitrogen to muscle mass, thereby reducing the amount of nitrogen that is available for production of ammonia and nitrous oxide.

In certain embodiments, the methods also reduce GHG emissions from the livestock animal's waste (e.g., urine and/or manure). In some embodiments, the beneficial microorganisms of the composition can survive transport through the digestive system and are excreted with the animal's waste, where they continue inhibiting methanogens and/or symbionts thereof, disrupting methanogenic biofilms, disrupting the biological pathways involved in methanogenesis, and/or compensating for H₂ acceptor loss. The composition can be administered to the livestock animal's digestive system and/or directly to the waste product.

In certain specific embodiments, the composition can be administered directly to a manure lagoon, waste pond, tailing pond, tank or other storage facility where livestock manure is stored and/or treated. Advantageously, in some embodiments, the microorganisms in the composition can facilitate an increased decomposition rate for the manure while reducing the amount of methane and/or nitrous oxide emitted therefrom. Furthermore, in some embodiments, applying the composition to the manure enhances the value of the manure as an organic fertilizer due to the ability of the microorganisms to inoculate the soil to which the manure is applied. The microbes then grow and, for example, improve soil biodiversity, enhance rhizosphere properties, and enhance plant growth and health.

In some embodiments, the methods can further comprise adding materials to enhance the growth of the microorganisms of the subject composition at the time of application (e.g., adding nutrients and/prebiotics). In certain embodiments, the livestock animal can be fed a source of prebiotics, which can include, for example, dry animal fodder, straw, hay, alfalfa, grains, forage, grass, fruits, vegetables, oats, and/or crop residue.

In some embodiments, the methods can be utilized for enhancing the overall health of a livestock animal, for example, by contributing to a healthy gut microbiome, improving digestion, increasing feed-to-muscle conversion ratio, increasing milk production and quality, reducing and/or treating dehydration and heat stress, modulating the immune system, and increasing life expectancy.

In some embodiments, the methods of the subject invention can be utilized by a livestock producer for reducing carbon credit usage. Thus, in certain embodiments, the subject methods can further comprise conducting measurements to assess the effect of the method on reducing the generation of methane, carbon dioxide and/or other deleterious atmospheric gases, and/or precursors thereof (e.g., nitrogen and/or ammonia), and/or to assess the effect of the method on the control of methanogens and/or protozoa in the livestock animal's digestive system and/or waste, using standard techniques in the art.

### BRIEF DESCRIPTION OF THE FIGURES

FIG. 1 shows biological pathways involved in methanogenesis.

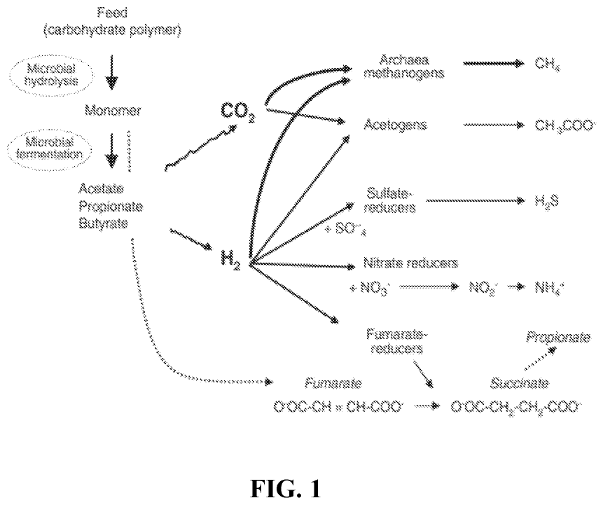

FIG. 2 shows the results of in-vitro studies of compositions according to embodiments of the subject invention to determine their ability to reduce enteric methane emissions from cattle rumen.

FIG. 3 shows the results of in-vitro studies of compositions according to embodiments of the subject invention to determine their ability to reduce enteric carbon dioxide emissions from cattle rumen.

FIG. 4 shows the results of in-vitro studies of B. amy at variable inclusion rates to determine its ability to reduce enteric methane emissions from cattle rumen.

FIG. 5 shows the results of in-vitro studies of B. amy at variable inclusion rates to determine its ability to reduce enteric carbon dioxide emissions from cattle rumen.

### DETAILED DESCRIPTION OF THE INVENTION

The subject invention provides compositions and methods for reducing atmospheric greenhouse gas emissions from livestock production. More specifically, the subject invention provides compositions that, when contacted with the digestive system and/or waste of a livestock animal, lead to a reduction in greenhouse gas emissions that would have otherwise been produced by the animal's digestive processes. Advantageously, in some embodiments, the compositions and methods can also improve the overall health and productivity of livestock animals.

In specific embodiments, the subject invention provides a digestive health composition for reducing methane, carbon dioxide, and/or other deleterious atmospheric gases and/or precursors thereof produced in a livestock animal's digestive system and/or waste, wherein the composition comprises one or more beneficial microorganisms and/or one or more microbial growth by-products.

### Selected Definitions

As used herein, a “biofilm” is a complex aggregate of microorganisms, such as bacteria, wherein the cells adhere to each other and/or to a surface. The cells in biofilms are physiologically distinct from planktonic cells of the same organism, which are single cells that can float or swim in liquid medium.

As used herein, the term “control” used in reference to an undesirable microorganism (e.g., a methanogen) extends to the act of killing, disabling, immobilizing and/or reducing the population numbers of the microorganism, and/or otherwise rendering the microorganism incapable of reproducing and/or carrying out the processes that are undesirable (e.g., methane production).

As used herein, the “digestive system” refers to the system of organs in an animal's body that enables digestion, or the consumption of food and conversion thereof to energy and waste. The digestive system can comprise, for example, an oral cavity, esophagus, crop, gizzard, proventriculus, stomach, rumen, reticulum, omasum, abomasum, pancreas, liver, small intestine, large intestine (colon), cecum, appendix, and/or anus. Additional organs or parts related to digestion and that are specific to a particular animal are also envisioned.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

As used herein, “ionophores” are carboxylic polyether non-therapeutic antibiotics that disrupt the ion concentration gradient (Ca2+, K+, H+, Na+) across microorganisms, which causes them to enter a futile ion cycle. The disruption of the ion concentration prevents the microorganism from maintaining normal metabolism and causes the microorganism to expend extra energy. Ionophores function by selecting against or negatively affecting the metabolism of gram-positive bacteria, such as methanogens, and protozoa.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, polyketides, and surfactants.

As used herein, a “methanogen” is a microorganism that produces methane gas as a by-product of metabolism. Methanogens are archaea that can be found in the digestive systems and metabolic waste of ruminant animals and non-ruminant animals (e.g., pigs, poultry and horses). Examples of methanogens include, but are not limited to, Methanobacterium spp. (e.g., M. formicicum), Methanobrevibacter spp. (e.g., M. ruminantium), Methanococcus spp. (e.g., M. paripaludis), Methanoculleus spp. (e.g., M. bourgensis), Methanoforens spp. (e.g., M. stordalenmirensis), Methanofollis liminatans, Methanogenium wolfei, Methanomicrobium spp. (e.g., M. mobile), Methanopyrus kandleri, Methanoregula boonei, Methanosaeta spp. (e.g., M. concilii, M. thermophile), Methanosarcina spp. (e.g., M. barkeri, M. mazeii), Methanosphaera stadimanae, Methanospirillium hungatei, Methanothermobacter spp., and/or Methanothrix sochngenii.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Digestive Health Compositions

In preferred embodiments, the subject invention provides a digestive health composition for reducing methane, carbon dioxide, nitrogen and/or other deleterious atmospheric gases and/or precursors thereof produced in a livestock animal's digestive system and/or waste, wherein the composition comprises one or more beneficial microorganisms and/or one or more microbial growth by-products.

In certain embodiments, the digestive health composition is a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The cells may be totally absent, or present at, for example, a concentration of 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter of the composition.

Advantageously, in preferred embodiments, the subject compositions can alter the digestive processes of livestock animals, resulting in decreased enteric atmospheric gas production.

In some embodiments, the subject compositions can be used for reducing methane, carbon dioxide and/or nitrous oxide production in livestock and/or livestock waste. For example, the compositions can directly inhibit or control methanogenic bacteria and/or symbionts thereof in the animal's digestive system and/or waste, as well as disrupt the integrity and/or production of biofilms formed by methanogens. Additionally, in some embodiments, the compositions can interfere with biological pathways involved in methanogenesis. Furthermore, in some embodiments, the compositions can compensate for a loss of H₂ acceptor compounds that results when methanogenesis is reduced.

In some embodiments, the composition can also enhance the growth and health of livestock, while enabling more complete transformation of protein sources in feed to reduce nitrogen release in the animals' waste in the form of, e.g., ammonia and/or urea. Advantageously, in some embodiments, this can result in reduced nitrous oxide production.

In preferred embodiments, the beneficial microorganisms of the subject compositions are non-pathogenic fungi, yeasts and/or bacteria. The beneficial microorganisms may be in an active, inactive and/or dormant. In preferred embodiments, the microorganism is one that is characterized as “generally regarded as safe,” or GRAS, by the appropriate regulatory agency.

The microorganisms of the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In some embodiments, the beneficial microorganisms are selected based on a natural or acquired resistance to certain antibiotics administered to a livestock animal to, for example, control pathogenic and/or deleterious microbes in the digestive system or elsewhere in the animal's body.

In some embodiments, the beneficial microorganisms of the subject composition are capable of surviving transport through the livestock animal's digestive system and are excreted in the animal's waste (e.g., manure). Thus, in certain embodiments, administering a composition according to embodiments of the subject invention to the animal can result in a reduction in GHG production in the animal's waste via inhibition of methanogens and/or symbionts thereof, disruption of methanogen biofilms, interference with biological pathways involved in methanogenesis, and compensation for H₂ acceptor loss.

In one specific embodiment, the composition comprises about 1×10⁶ to about 1×10¹³, about 1×10⁷ to about 1×10¹², about 1×10⁸ to about 1×10¹¹, or about 1×10⁹ to about 1×10¹⁰ CFU/g of each species of microorganism present in the composition.

In one embodiment, the composition comprises about 1 to 100% microorganisms total by volume, about 10 to 90%, or about 20 to 75%.

In an exemplary embodiment, the amount of microorganisms in one application of the composition totals about 1 to 100 grams per head (individual animals in a herd or flock), or about 5 to about 85 grams per head, or about 10 to about 70 grams per head, or about 15 to 50 grams per head.

In certain preferred embodiments, the composition comprises one or more bacteria and/or growth by products thereof. The bacteria can be, for example, a Myxococcus sp. (e.g., M. xanthus), and/or one or more Bacillus spp. bacteria. In certain embodiments, the Bacillus spp. are B. amyloliquefaciens, B. subtilis (e.g., strain “B4”) and/or B. licheniformis. Bacteria can be used in spore form, as vegetative cells, and/or as a mixture thereof.

In one embodiment, the composition comprises B. amyloliquefaciens. In a preferred embodiment, the strain of B. amyloliquefaciens is B. amyloliquefaciens NRRL B-67928 (“B. amy”).

A culture of the B. amyloliquefaciens “B. amy” microbe has been deposited with the Agricultural Research Service Northern Regional Research Laboratory (NRRL), 1400 Independence Ave., S.W., Washington, DC, 20250, USA. The deposit has been assigned accession number NRRL B-67928 by the depository and was deposited on Feb. 26, 2020.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

In one embodiment, the beneficial microorganisms are yeasts and/or fungi. Yeast and fungus species suitable for use according to the current invention, include Acaulospora, Acrenionium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T. viridae), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bailii), and others.

In certain specific embodiments, the composition comprises one or more fungi and/or one or more growth by-products thereof. The fungi can be, for example, Pleurotus spp. fungi, e.g., P. ostreatus (oyster mushrooms), Lentinula spp. fungi, e.g., L. edodes (shiitake mushrooms), and/or Trichoderma spp. fungi, e.g., T. viridae. The fungi can be in the form of live or inactive cells, mycelia, spores and/or fruiting bodies. The fruiting bodies, if present, can be, for example, chopped and/or blended into granules and/or a powder form.

In certain specific embodiments, the composition comprises one or more yeasts and/or one or more growth by-products thereof. The yeast(s) can be, for example, Wickerhamomyces anomalus, Saccharomyces spp. (e.g., S. cerevisiae and/or S. boulardii), Debaryomyces hansenii, Starmerella bombicola, Meyerozyma guilliermondii, Pichia occidentalis, Monascus purpureus, and/or Acremonium chrysogenum. The yeast(s) can be in the form of live or inactive cells or spores, as well as in the form of dried and/or dormant cells (e.g., a yeast hydrolysate).

In one exemplary embodiment, the composition comprises a strain of B. amyloliquefaciens, e.g., B. amy. In another exemplary embodiment, the composition comprises P. ostreatus and S. boulardii. In another exemplary embodiment, the composition comprises D. hansenii. In yet another exemplary embodiment, the composition comprises B. amy, P. ostreatus, S. boulardii or D. hansenii, and/or any combination thereof.

In one embodiment, the microbe-based composition comprises a microbial growth by-product. The microbial growth by-product can be produced by the microorganisms of the composition, and/or they can be produced separately and added to the composition.

In one embodiment, the growth by-product has been purified from the cultivation medium in which it was produced. Alternatively, in one embodiment, the growth by-product is utilized in crude form. The crude form can comprise, for example, a liquid supernatant resulting from cultivation of a microbe that produces the growth by-product of interest, including residual cells and/or nutrients.

The growth by-products can include metabolites or other biochemicals produced as a result of cell growth, including, for example, amino acids, peptides, polyketides, antibiotics, proteins, enzymes, biosurfactants, solvents, vitamins, and/or other metabolites.

The microorganism(s) and/or growth by-product(s) present in the composition can be useful for inhibiting methanogens and/or the methanogenesis pathway, disrupting methanogen biofilms, and/or reducing H₂ accumulation in a livestock animal's digestive system. Furthermore, in preferred embodiments, the composition can be useful for enhancing the overall health of a livestock animal. Bacillus spp.

In certain embodiments, the composition comprises B. amy and/or growth by-products thereof. B. amy is particularly advantageous over traditional probiotic microorganisms due to its ability to produce spores that remain viable in the digestive tract and, in some embodiments, after excretion in the animal's waste. Additionally, B. amy produces a unique mixture of metabolites that provide a broad-spectrum of digestive and environmental benefits when administered to a livestock animal and/or its waste.

In certain embodiments, as exemplified in Table 1 below, the growth by-products can directly inhibit methanogens, disrupt methanogen biofilms, and/or reduce H₂ concentration in a livestock animal's digestive system.

TABLE 1
Exemplary B. amy growth by-products
for reducing methanogenesis and H₂

| | Growth by- | |
|---|---|---|
| Function(s) | product(s) | Examples (Produced by B. amy) |
| Inhibition of | Enzymes | Proteinase K (and/or a homolog thereof): |
| methanogens | | can specifically lyse pseudomurien, a |
| | | major structural cell wall component of |
| | | some archaea, including methanogens. |
| | | Diglycolic acid dehydrogenase (DGADH), |
| | | (and/or a homolog thereof): can disrupt |
| | | ether bonds between the glycerol |
| | | backbone and fatty acids of the phospho- |
| | | lipid layer of archaeal cell membranes. |
| | Organic | Propionic acid and/or acetic acid: can |
| | acids | disrupt the structure of archaeal cell |
| | | membranes. |
| Disruption of | Lipopeptide | Surfactin, fengycin, iturin, bacillomycin, |
| methanogen | biosurfactants; | lichenysin, difficidin, and/or a maltose- |
| biofilms | Glycolipid | based glycolipid: can interfere with the |
| | biosurfactants; | production and/or maintenance of the |
| | Polyketides | exopolysaccharide matrix that forms |
| | | biofilms, thereby interfering with |
| | | formation and/or adhesion of |
| | | capabilities of the biofilm. |
| Reduction | Organic | Propionic acid: can stimulate acetogenic |
| of H₂ | acids | microorganisms, which produce acetic |
| | | acid from hydrogen and carbon dioxide. |
| | | This results in reduced hydrogen |
| | | availability for methanogenic microbes |
| | | to carry out methanogenesis, and also |
| | | helps keep H₂ concentrations |
| | | from increasing when methanogenesis |
| | | decreases. Increased H₂ can |
| | | lead to a build-up of trimethylamine |
| | | in the digestive system, which causes a |
| | | “fishy” smell in produced milk. |

In one embodiment, as exemplified in Table 2 below, the composition comprises B. amy, and/or growth by-products thereof, which can enhance the overall health and productivity of a livestock animal by performing a variety of health-promoting functions. Thus, in some embodiments, B. amy can serve as a probiotic when administered to an animal.

TABLE 2
Exemplary B. amy growth by-products for enhancing livestock health

| | Growth by- | |
|---|---|---|
| Function(s) | product(s) | Examples (Produced by B. amy) |
| Regulation of gut | Biosurfactants; | Biosurfactants, including lipopeptides and |
| microbiome | Natural antibiotics; | glycolipids, as well as natural antibiotics |
| | Organic acids | (e.g., polyketides, penicillins, |
| | | cephalosporins, validamycins, carbapanems, |
| | | and nocardicins): can inhibit the growth of |
| | | pathogenic, or otherwise deleterious gut |
| | | microorganisms (e.g., Anaeroplasma, |
| | | Acholeplasma and certain fungi) by, for |
| | | example, interfering with the pathogenic or |
| | | deleterious microorganism's cell membrane |
| | | and/or biofilm structure. |
| | | Organic acids, such as propionic acid: can |
| | | promote the growth of beneficial gut |
| | | microorganisms (e.g., Proteobacteria, |
| | | Rhodospirillaceae, Campylobacterales and |
| | | Butyricimonas) by, for example, altering the |
| | | pH of the digestive system to a more |
| | | favorable environment for such growth. |
| | | In certain embodiments, regulation of the |
| | | gut microbiome also leads to a reduction in |
| | | nitrous oxide emissions due to a reduction in |
| | | ammonia-oxidizing gut bacteria. |
| Stimulation of growth | Organic acids; | Organic acids, such as the short-chain fatty |
| hormones (e.g., GH/IGH-1); | Biosurfactants; | acids butyrate and valerate: can improve |
| increasing the rate of weight | Digestive enzymes | digestion through, for example, improved |
| gain; and increasing feed-to | | intestinal and/or ruminal cell function. |
| muscle conversion through | | Lipopeptide and glycolipid biosurfactants: |
| improved digestion | | can improve digestion by, for example, |
| | | enhancing the bioavailability of nutrients |
| | | and water through intestinal/ruminal cells |
| | | and improve absorption thereof into the |
| | | bloodstream. |
| | | Digestive enzymes, such as amylases, |
| | | lipases, and proteases (e.g., collagenase-like |
| | | protease, peptidase E (N-terminal Asp- |
| | | specific dipeptidase), peptidase s8 |
| | | (subtilisin-like serine peptidase), serine |
| | | peptidase, and endopeptidase La): can |
| | | improve conversion of feed to muscle by |
| | | increasing digestion of proteins, fats and |
| | | carbohydrates in feed that can otherwise be |
| | | difficult or impossible for the animal to |
| | | digest. |
| | | Additionally, because nitrogen is required |
| | | for conversion of feed to muscle mass, |
| | | increased nitrogen uptake in the digestive |
| | | system due to improved muscle conversion |
| | | can result in fewer nitrous oxide precursors, |
| | | and accordingly, fewer nitrous oxide |
| | | emissions. |
| Improving quantity and | Lignocellulytic | Lignocellulytic enzymes, such as cellulose, |
| quality of produced milk in | enzymes; | xylanase, laccase, and manganese catalase: |
| mammals | Folic acid/folate | can enhance digestion of polysaccharides, |
| | | such as cellulose, xylan, hemicellulose, and |
| | | lignin, into the components necessary for |
| | | milk production. |
| | | Folate: can help increase milk production |
| | | by, for example, enhancing mammary gland |
| | | metabolism. Additionally, folate is an |
| | | important nutrient for, e.g., growth and |
| | | neural development. Thus, increased folate |
| | | in produced milk can improve the nutritional |
| | | quality of the milk for nursing offspring, |
| | | thereby potentially shortening the time |
| | | required for weaning and/or increasing the |
| | | growth and survival rate of offspring. |
| Enhancing immune health, | Vitamins | Riboflavin, produced via riboflavin |
| life expectancy and overall | | synthase: can provide antinociception and |
| health | | anti-inflammatory effects in a livestock |
| | | animal. |
| | | Folate, produced via bifunctional folate |
| | | synthesis protein: can help regulate energy |
| | | conversion, gene expression and DNA |
| | | production, in addition to being an anti- |
| | | inflammatory agent. |
| | | Ubiquinone (CoQ10), produced via |
| | | ubiquinone biosynthesis O- |
| | | methyltransferase: can, as an antioxidant, |
| | | prevent low-density lipoprotein oxidation, |
| | | which can result in atherosclerosis. |

In some embodiments, the composition can comprise other species of Bacillus, such as, for example, B. licheniformis and/or B. subtilis. In some embodiments, B. licheniformis can reduce methane production by methanogens, and inhibit the methanogenic bacteria themselves through production of propionic acid and other metabolites, such as lipopeptide biosurfactants. Additionally, B. licheniformis can help decrease the concentration of ammonia in cattle ruminal fluids while helping increase milk protein production. In pigs, B. licheniformis and B. subtilis can help increase fecal Lactobacillus counts, increase the digestibility of nitrogen, and a decrease the emission of ammonia and mercaptans.

Pleurotus ostreatus

In certain embodiments, as exemplified in Table 3 below, the composition comprises P. ostreatus and/or growth by-products thereof, wherein the growth by-products can directly inhibit methanogens, disrupt methanogen biofilms, and/or reduce H₂ concentration in a livestock animal's digestive system.

TABLE 3
Exemplary P. ostreatus growth by-products
for reduction of methanogenesis

| | Growth by- | |
|---|---|---|
| Function(s) | product(s) | Examples (Produced by P. ostreatus) |
| Inhibition of | Polyketides | Lovastatin, and/or a homologous |
| methanogens | | polyketide thereof: can inhibit HMG-CoA |
| | | reductase, the enzyme involved in |
| | | formation of the isoprenoid building |
| | | blocks that are essential for archaea cell |
| | | membrane synthesis. Advantageously, |
| | | lovastatin can inhibit the growth of |
| | | methanogens without adverse effects on |
| | | other celluloytic bacteria in the digestive |
| | | system. |
| Disruption of | Lipopeptide | Lipopeptide biosurfactants, such as |
| methanogen | biosurfactants | surfactins, iturins, fengycins, and |
| biofilms | | lichenysins: can interfere with the |
| | | production and/or maintenance of the |
| | | exopolysaccharide matrix that forms |
| | | biofilms, thereby interfering with |
| | | formation and/or adhesion of capabilities |
| | | of the biofilm. |

In certain embodiments, a composition comprising P. ostreatus must be supplemented with an H₂ acceptor to reduce H₂ buildup resulting from decreased methanogenesis. For example, in some embodiments, B. amy, Saccharomyces boulardii and/or Debaryomyces hansenii can be included to supply H₂ acceptors to the digestive system.

In one embodiment, as exemplified in Table 4 below, the composition also comprises P. ostreatus growth by-products that can enhance the overall health and productivity of a livestock animal by performing a variety of health-promoting functions. Thus, in some embodiments, P. ostreatus can serve as a probiotic when administered to an animal.

TABLE 4
Exemplary P. ostreatus growth by-products for enhancing livestock health

| Function(s) | Growth by-product(s) | Examples (Produced by P. ostreatus) |
|---|---|---|
| Regulation of gut | Biosurfactants; | Lipopeptide biosurfactants, as well as |
| microbiome | Natural antibiotics; | natural antibiotics (e.g., pleurotin, |
| | Short chain fatty acids | leucopleurotin, and dihydropleurotinic acid): |
| | | can inhibit the growth of pathogenic, or |
| | | otherwise deleterious gut microorganisms |
| | | by, for example, interfering with the |
| | | pathogenic or deleterious microorganism's |
| | | cell membrane and/or biofilm structure. |
| | | Short chain fatty acids, such as linoleic acid |
| | | and S-coriolic acid: can be toxic to certain |
| | | nematode species, and can inhibit the |
| | | growth of pathogenic bacteria. |
| | | Other secondary metabolites of P. ostreatus |
| | | can also have inhibitory activity against |
| | | detrimental. yeast species such as C. |
| | | albicans, P. aeruginosa, and S. aureus. |
| Improving growth rate and | Biosurfactants; | Biosurfactants, including lipopeptides: can |
| efficiency of digestion, | Short chain fatty acids; | improve digestion by, for example, |
| including reducing | Lignocellulytic enzymes; | enhancing the bioavailability of nutrients |
| diarrhea | Nutrients | and water through intestinal/ruminal cells |
| | | and improve absorption thereof into the |
| | | bloodstream. |
| | | Short chain fatty acids, such as linoleic acid |
| | | and S-coriolic acid: can increase the |
| | | absorption of minerals in the digestive |
| | | system. |
| | | Lignocellulytic enzymes, such as cellulose, |
| | | xylanase, laccase, and manganese |
| | | peroxidase: can enhance digestion of |
| | | polysaccharides, such as cellulose, xylan, |
| | | hemicellulose, and lignin. |
| | | Pleurotus ostreatus is a source of growth- |
| | | promoting nutrients, including selenium and |
| | | protein. Pleurotus ostreatus comprises 30- |
| | | 35% protein by weight, and about 55 to |
| | | 60 ug/g of selenium. Selenium is an essential |
| | | trace mineral for cattle, involved in proper |
| | | immune function, acts as an antioxidant, and |
| | | helps activate thyroid hormones. Selenium |
| | | deficiency causes muscle damage, increase |
| | | rate of illness, impaired growth and |
| | | decreased reproductive efficiency. |
| Improving quantity and | Lignocellulytic | Lignocellulytic enzymes, such as cellulose, |
| quality of produced milk in | enzymes | xylanase, laccase, and manganese catalase, |
| mammals | | can enhance digestion of polysaccharides, |
| | | such as cellulose, xylan, hemicellulose, and |
| | | lignin, into the components necessary for |
| | | milk production. |

Saccharomyces boulardii

In certain embodiments, as exemplified in Table 5 below, the composition comprises S. boulardii and/or growth by-products thereof, wherein the growth by-products can directly inhibit methanogens, disrupt methanogen biofilms, and/or reduce H₂ concentration in a livestock animal's digestive system.

TABLE 5
Exemplary S. boulardii growth by-products
for reduction of methanogenesis and H₂

| Function(s) | Growth by-product(s) | Examples (Produced by S. boulardii) |
|---|---|---|
| Inhibition of | Enzymes | Proteinase K (and/or a homolog thereof): |
| methanogens | | can specifically lyse pseudomurien, a |
| | | major structural cell wall component of |
| | | some archaea, including methanogens. |
| Reduction | Digestive enzymes; | Digestive enzymes, including sucrose and |
| of H₂ | Vitamins; | isomaltase: sucrose can catalyze the |
| | Organic acids | hydrolysis of sucrose to fructose and |
| | | glucose, while isomaltase can digest |
| | | starch and other polysaccharides. |
| | | Hydrogen is released from malabsorbed |
| | | sucrose and starch by ruminal microflora. |
| | | B vitamins and organic acids: can |
| | | stimulate acetogenic microorganisms, |
| | | which produce acetic acid from hydrogen |
| | | and carbon dioxide. This results in |
| | | reduced hydrogen availability for |
| | | methanogenic microbes to carry out |
| | | methanogenesis, and also helps keep H₂ |
| | | concentrations from increasing when |
| | | methanogenesis decreases. |

In one embodiment, as exemplified in Table 6 below, the composition also comprises S. boulardii growth by-products that can enhance the overall health and productivity of a livestock animal by performing a variety of health-promoting functions. Thus, in some embodiments, S. boulardii can serve as a probiotic when administered to an animal.

TABLE 6
Exemplary S. boulardii growth by-products for enhancing livestock health

| | Growth by- | |
|---|---|---|
| Function(s) | product(s) | Examples (Produced by S. pleurotus) |
| Regulation of gut | Terpenes and | Sesquiterpenoids and triterpenoids: can |
| microbiome | terpenoids; | reduce the negative effects of pathogens |
| | Natural | (e.g., E. coli, Vibrio cholera, Clostridium |
| | antibiotics; | difficile, and Salmonella spp.), such as |
| | Fatty acids; | gastroenteritis. |
| | Enzymes | Natural antibiotics (e.g., polyketides, |
| | | penicillins, cephalosporins, validamycins, |
| | | carbapanems, and nocardicins): can inhibit |
| | | the growth of pathogenic, or otherwise |
| | | deleterious gut microorganisms by, for |
| | | example, interfering with the pathogenic or |
| | | deleterious microorganism's cell membrane |
| | | and/or biofilm structure. |
| | | Fatty acids, such as pyruvate, B-alanine, |
| | | lipoic acid, decanoic acid, and capric acid: |
| | | can inhibit adhesion and/or biofilm |
| | | formation by Candida albicans. |
| | | Enzymes, such as a 54 kDa protease: can |
| | | degrade Clostridial toxins. |
| | | Pantothenate (vitamin B5): can reduce |
| | | bacterial growth by regulating innate |
| | | immunity and adaptive immunity. |
| Improving growth | Vitamins; | B vitamins and organic acids: can stimulate |
| rate and efficiency | Organic acids; | acetogenic microorganisms, which produce |
| of digestion | Amino acids | acetic acid, a source of carbon, from |
| | | hydrogen and carbon dioxide. |
| | | Valine, an amino acid: enables more |
| | | complete transformation of protein sources |
| | | in feed. Additionally, enhanced feed |
| | | transformation reduces the amount of |
| | | nitrogen excreted in waste, in the form of, |
| | | for example, ammonia, thereby reducing |
| | | NO₂ precursors. |
| Improving quantity and | Folate/folic | Folate: can help increase milk production |
| quality of produced milk | acid | by, for example, enhancing mammary gland |
| in mammals | | metabolism. Additionally, folate is an |
| | | important nutrient for, e.g., growth and |
| | | neural development. Thus, increased folate |
| | | in produced milk can improve the nutritional |
| | | quality of the milk for nursing offspring, |
| | | thereby potentially shortening the time |
| | | required for weaning and/or increasing the |
| | | growth and survival rate of offspring. |
| Enhancing immune health, | Terpenes and | Sesquiterpenoids and triterpenoids: can |
| life expectancy and overall | terpenoids; | reduce the negative effects of pathogens |
| health | Natural | (e.g., E. coli, Vibrio cholera, Clostridium |
| | antibiotics; | difficile, and Salmonella spp.), such as |
| | Fatty acids; | gastroenteritis. |
| | Enzymes; | Natural antibiotics (e.g., polyketides, |
| | Vitamins | penicillins, cephalosporins, validamycins, |
| | | carbapanems, and nocardicins): can inhibit |
| | | the growth of pathogenic, or otherwise |
| | | deleterious gut microorganisms by, for |
| | | example, interfering with the pathogenic or |
| | | deleterious microorganism's cell membrane |
| | | and/or biofilm structure. |
| | | Fatty acids, such as pyruvate, B-alanine, |
| | | lipoic acid, decanoic acid, and capric acid: |
| | | can inhibit adhesion and/or biofilm |
| | | formation by Candida albicans. |
| | | Enzymes, such as a 54 kDa protease: can |
| | | degrade Clostridial toxins. |
| | | Pantothenate (vitamin B5), produced via |
| | | panthothenate synthase: can reduce bacterial |
| | | growth by regulating innate immunity and |
| | | adaptive immunity. |
| | | Riboflavin, produced via riboflavin |
| | | synthase: can provide antinociception and |
| | | anti-inflammatory effects in a livestock |
| | | animal. |
| | | Folate, produced via FOL1p multifunctional |
| | | enzyme: can help regulate energy |
| | | conversion, gene expression and DNA |
| | | production, in addition to being an anti- |
| | | inflammatory agent. |
| | | Ubiquinone (CoQ10), produced via |
| | | ubiquinone biosynthesis protein CoQ9p: can |
| | | enhance respiratory health and, as an |
| | | antioxidant, prevent low-density lipoprotein |
| | | oxidation, which can result in |
| | | atherosclerosis. |
| | | Secondary metabolites: can reduce a pro- |
| | | inflammatory state by decreasing the levels |
| | | of CD40−, CD80−, and CD197 and reducing |
| | | their secretion of tumor necrosis factor |
| | | (TNF)-α and interleukin (IL)-6. |

Debaryomyces hansenii

In certain embodiments, as exemplified in Table 7 below, the composition comprises D. hansenii and/or growth by-products thereof, wherein the growth by-products can directly inhibit methanogens, disrupt methanogen biofilms, and/or reduce H₂ concentration in a livestock animal's digestive system.

TABLE 7
Exemplary D. hansenii growth by-products
for reduction of methanogenesis and H₂

| | Growth by- | |
|---|---|---|
| Function(s) | product(s) | Examples (Produced by D. hanseniii) |
| Inhibition of | Enzymes; | Proteinase K (and/or a homolog thereof): |
| methanogens | Fatty acids; | can specifically lyse pseudomurien, a |
| | Organic acids | major structural cell wall component of |
| | | some archaea, including methanogens. |
| | | Medium chain fatty acids, such as |
| | | palmitic acid: can be directly toxic to |
| | | methanogens. Fatty acid production is |
| | | enhanced in extreme conditions, such as |
| | | low pH and high temperature. |
| | | Propionic acid: can stimulate acetogenic |
| | | microorganisms, which produce acetic |
| | | acid from hydrogen and carbon dioxide. |
| | | This results in reduced hydrogen |
| | | availability for methanogenic microbes to |
| | | carry out methanogenesis. |
| Disruption of | Glycolipid | Glycolipid biosurfactants, such as |
| methanogen | biosurfactants | sophorolipids: can interfere with the |
| biofilm | | production and/or maintenance of the |
| | | exopolysaccharide matrix that forms |
| | | biofilms, thereby interfering with |
| | | formation and/or adhesion of capabilities |
| | | of the biofilm. |
| Reduction | Organic acids | Organic acids, such as acetic acid, pyruvic |
| of H₂ | | acid, citric acid, and formic acid: can |
| | | inhibit methanogenesis and reduce H₂ |
| | | This results in reduced hydrogen |
| | | availability for methanogenic microbes to |
| | | carry out methanogenesis, and also helps |
| | | keep H₂ concentrations from increasing |
| | | when methanogenesis decreases. |

In one embodiment, as exemplified in Table 8 below, the composition also comprises D. hansenii growth by-products that can enhance the overall health and productivity of a livestock animal by performing a variety of health-promoting functions. Thus, in some embodiments, D. hansenii can serve as a probiotic when administered to an animal.

TABLE 8
Exemplary D. hansenii growth by-products for enhancing livestock health

| Function(s) | Growth by-product(s) | Examples (Produced by D. hansenii) |
|---|---|---|
| Regulation of gut | Aldehydes; | Aldehydes such as 2-methylpropanal and 3- |
| microbiome | Natural | methylbutanal: can have antibacterial and |
| | antibiotics | antioxidant activity. |
| | | Natural antibiotics, such as a 22 kDa |
| | | mycocin: can have activity against |
| | | Staphylococcus aureus, Escherichia coli, |
| | | Klebsiella pneumoniae, Streptococcus |
| | | pyogenes, Candida albicans, and Candida |
| | | neoformans |
| Improving growth | Glycolipids; | Glycolipid biosurfactants, such as |
| rate and efficiency | Digestive enzymes; | sophorolipids: can improve digestion by, for |
| of digestion | Phosphatidylinositol; | example, enhancing the bioavailability of |
| | Vitamins | nutrients and water through |
| | | intestinal/ruminal cells and improve |
| | | absorption thereof into the bloodstream. |
| | | Digestive enzymes, such as amylases, |
| | | lipases, and proteases (e.g., Protease A, B, |
| | | D): can improve conversion of feed to |
| | | muscle by increasing digestion of proteins, |
| | | fats and carbohydrates in feed that can |
| | | otherwise be difficult or impossible for the |
| | | animal to digest. |
| | | Phosphatidylinositol: can increase |
| | | sensitivity to insulin. |
| | | Ergosterol: can serve as a vitamin D |
| | | precursor, which plays a vital role in |
| | | immune and growth processes, and |
| | | improves calcium reabsorption. |
| Improving quantity | Folate/folic | Folate: can help increase milk production |
| and quality of | acid; | by, for example, enhancing mammary gland |
| produced milk in | | metabolism. Additionally, folate is an |
| mammals | | important nutrient for, e.g., growth and |
| | | neural development. Thus, increased folate |
| | | in produced milk can improve the |
| | | nutritional quality of the milk for nursing |
| | | offspring, thereby potentially shortening the |
| | | time required for weaning and/or increasing |
| | | the growth and survival rate of offspring. |
| Enhancing immune health, | Vitamins; | Folate: can help regulate energy conversion, |
| life expectancy | Amines; | gene expression and DNA production, in |
| and overall | Phosphatidylinositol | addition to being an anti-inflammatory |
| health | | agent. |
| | | Riboflavin: can provide antinociception and |
| | | anti-inflammatory effects in a livestock |
| | | animal. |
| | | Ergosterol: can serve as a vitamin D |
| | | precursor, which plays a vital role in |
| | | immune and processes, and |
| | | improves calcium reabsorption. |
| | | Amines, such as spermidine and spermine: |
| | | can synchronize an array of biological (such |
| | | as Ca²⁺, Na⁺, K⁺ -ATPase), thus maintaining |
| | | membrane potential and controlling |
| | | intracellular pH and volume. Spermidine |
| | | regulates biological processes, such as |
| | | Ca²⁺ influx by glutamatergic N-methyl-d- |
| | | aspartate receptor (NMDA receptor), which |
| | | has been associated with nitric oxide |
| | | synthase (NOS) and cGMP/PKG pathway |
| | | activation and a decrease of Na+, K+- |
| | | ATPase activity in cerebral cortex |
| | | synaptosomes. Spermidine is |
| | | a longevity agent in mammals due to |
| | | various mechanisms of action. Autophagy is |
| | | the main mechanism at the molecular level, |
| | | but evidence has been found for other |
| | | mechanisms, including inflammation |
| | | reduction, lipid metabolism, and regulation |
| | | of cell growth, proliferation and death |
| | | Phosphatidylinositol: can enhance fertility, |
| | | reduce oxidate stress, raise plasma HDL- |
| | | cholesterol and apolipoprotein A-1 levels, |
| | | and reduce triglyceride levels. |
| | | D. hansenii has the ability to survive GI |
| | | stresses, for example, due to heat tolerance |
| | | activated by phosphatidylinositol. It can also |
| | | adhere to Caco-2 cells and mucin and |
| | | induce a high IL-10/IL-12 ratio, which can |
| | | have an anti-inflammatory effect. D. |
| | | hansenii can also stimulate innate immune |
| | | and antioxidant parameters and the |
| | | expression of immune-related gene |
| | | signaling pathways |

### Other Microorganisms

In certain embodiments, the composition can comprise one or more other microorganisms and/or growth by-products thereof that are useful for reducing methane production and/or enhancing livestock health.

In one embodiment, the composition comprises live Lentinula edodes, which can inhibit HMG-CoA reductase activity without production of lovastatin.

In one embodiment, the composition comprises Trichoderma viridae and/or Acremonium chrysogenum, which also produce statins similar to lovastatin with potential to act as HMG-CoA reductase inhibitors.

In one embodiment, the composition comprises red yeast rice, or koji, the fermented rice product of Monascus purpureus. Red yeast rice comprises monacolin K, which has a similar structure to lovastatin and has the ability to inhibit HMG-CoA reductase activity.

In one embodiment, the composition comprises Wickerhamomyces anomalus yeasts, which can boost acetogenesis and hydrogen utilization by acetogenic bacteria within a ruminant digestive system, thereby reducing methanogenesis and/or crowding out methanogenic bacteria. Advantageously, this results in less hydrogen availability for methanogenic microorganism to carry out processes in which methane is produced, without negatively affecting the digestive health of the animal.

Additionally, Wickerhamomyces anomalus produces phytase, an enzyme useful for improved digestion and bioavailability of phosphorus from feed, as well as killer toxins (e.g., exo-β-1,3-glucanase) useful for controlling pathogenic microorganisms.

Furthermore, Wickerhamomyces anomalus produces valine, an amino acid that helps support the growth and health of livestock animals, and enables more complete transformation of protein sources in feed to reduce the amount of nitrogen excreted in their waste, in the form of, for example, ammonia.

### Additional Components

In certain embodiments, the composition comprises a germination enhancer for enhancing germination of spore-form microorganisms used in the microbe-based composition. In specific embodiments, the germination enhancers are amino acids, such as, for example, L-alanine and/or L-leucine. In one embodiment, the germination enhancer is manganese.

In one embodiment, the composition comprises one or more fatty acids. The fatty acids can be produced by the microorganisms of the composition, and/or produced separately and included as an additional component. In certain preferred embodiments, the fatty acid is a saturated long-chain fatty acid, having a carbon backbone of 14-20 carbons, such as, for example, myristic acid, palmitic acid or stearic acid. In some embodiments, a combination of two or more saturated long-chain fatty acids is included in the composition. In some embodiments, a saturated long-chain fatty acid can inhibit methanogenesis and/or increase cell membrane permeability of methanogens.

In some embodiments, the composition can comprise additional components known to reduce methane in the livestock animal's digestive system, such as, for example, seaweed (e.g., Asparagopsis taxiformis and/or Asparagopsis armata); kelp; nitrooxypropanols (e.g., 3-nitrooxypropanol and/or ethyl-3-nitrooxypropanol); anthraquinones; ionophores (e.g., monensin and/or lasalocid); polyphenols (e.g., saponins, tannins); Yucca schidigera extract (steroidal saponin-producing plant species); Quillaja saponaria extract (triterpenoid saponin-producing plant species); organosulfurs (e.g., garlic extract); flavonoids (e.g., quercetin, rutin, kaemferol, naringin, and anthocyanidins; bioflavonoids from green citrus fruits, rose hips and black currants); carboxylic acid; and/or terpenes (e.g., d-limonene, pinene and citrus extracts).

In a specific exemplary embodiment, the composition comprises a microorganism, e.g., B. amy, and 3-nitrooxypropanol (3NOP), an organic compound having the formula HOCH₂CH₂CH₂ONO₂. 3NOP is effective for suppressing one or more enzymes involved in methanogenesis, e.g., methyl coenzyme M reductase (Mcr).

Mcr mediates the final step of all methanogenesis pathways, with CoM (2-mercaptoethanesulfonic acid) as an essential co-factor serving as the methyl group carrier. Mcr reduces methyl-CoM to methane. 3NOP can competitively bind to the Mer active site and then oxidize the Ni¹⁺ that is required for Mcr activity. (Patra et al. 2017).

In some embodiments, including 3NOP in the subject composition can result in inactivation or inhibition of Mcr and thus, reduced methane emissions from livestock.

In one embodiment, the subject composition can comprise one or more additional substances and/or nutrients to supplement the livestock animal's nutritional needs and promote health and/or well-being in the livestock animal, such as, for example, sources of amino acids (including essential amino acids), peptides, proteins, vitamins, microelements, fats, fatty acids, lipids, carbohydrates, sterols, enzymes, and minerals such as calcium, magnesium, phosphorus, potassium, sodium, chlorine, sulfur, chromium, cobalt, copper, iodine, iron, manganese, molybdenum, nickel, selenium, and zinc. In some embodiments, the microorganisms of the composition produce and/or provide these substances.

In one embodiment, the composition can comprise one or more biosurfactants. Biosurfactants are a structurally diverse group of surface-active substances produced by microorganisms, which are biodegradable and can be efficiently produced using selected organisms on renewable substrates. All biosurfactants are amphiphiles. They consist of two parts: a polar (hydrophilic) moiety and non-polar (hydrophobic) group. The common lipophilic moiety of a biosurfactant molecule is the hydrocarbon chain of a fatty acid, whereas the hydrophilic part is formed by ester or alcohol groups of neutral lipids, by a carboxylate group of fatty acids or amino acids (or peptides), an organic acid in the case of flavolipids, or, in the case of glycolipids, by a carbohydrate.

Due to their amphiphilic structure, biosurfactants increase the surface area of hydrophobic water-insoluble substances, increase the water bioavailability of such substances, and change the properties of bacterial cell surfaces. Biosurfactants accumulate at interfaces, thus reducing interfacial tension and leading to the formation of aggregated micellar structures in solution. Safe, effective microbial biosurfactants reduce the surface and interfacial tensions between the molecules of liquids, solids, and gases. The ability of biosurfactants to form pores and destabilize biological membranes permits their use as antibacterial, antifungal, and hemolytic agents.

Biosurfactants according to the subject invention can include, for example, glycolipids, lipopeptides, flavolipids, phospholipids, fatty acid esters, and high molecular weight polymers such as lipoproteins, lipopolysaccharide-protein complexes, and polysaccharide-protein-fatty acid complexes.

In one embodiment, the biosurfactant is a glycolipid. Glycolipids can include, for example, sophorolipids, rhamnolipids, cellobiose lipids, mannosylerythritol lipids and trehalose lipids. In one embodiment, the biosurfactant is a lipopeptide. Lipopeptides can include, for example, surfactin, iturin, arthrofactin, viscosin, fengycin, and lichenysin. In certain embodiments, a mixture of biosurfactants is used.

In one embodiment, the biosurfactant has been purified from the fermentation medium in which it was produced. Alternatively, in one embodiment, the biosurfactant is utilized in crude form comprising fermentation broth resulting from cultivation of a biosurfactant-producing microbe. This crude form biosurfactant solution can comprise from about 0.001% to 99%, from about 25% to about 75%, from about 30% to about 70%, from about 35% to about 65%, from about 40% to about 60%, from about 45% to about 55%, or about 50% pure biosurfactant, along with residual cells and/or nutrients.

In one embodiment, the composition comprises a saponin at 1 to 10 ml/L, or 2 to 6 ml/L of ruminal fluid. Saponins are natural surfactants that are found in many plants and that exhibit similar characteristics to microbial biosurfactants, for example, self-association and interaction with biological membranes. There are three basic categories of saponins, including triterpenoid saponins, steroidal saponins, and steroidal glycoalkaloids.

Some well-known triterpenoid saponin-accumulating plant families include the Leguminosae, Amaranthaceae, Apiaceae, Caryophyllaceae, Aquifoliaceae, Araliaceae, Cucurbitaceae, Berberidaceae, Chenopodiaceae, Myrsinaceae and Zygophyllaceae, among many others. Quillaja and legumes such as soybeans, beans and peas are a rich source of triterpenoid saponins. The steroidal saponins are typically found in members of the Agavaceae, Alliaceae, Asparagaceae, Dioscoreaceae, Liliaceae, Amaryllidaceae, Bromeliaceae, Palmae and Scrophulariaceae families and accumulate in abundance in crop plants such as yam, alliums, asparagus, fenugreek, yucca and ginseng. The steroidal glycoalkaloids are commonly found in members of the Solanaceae family including tomato, potato, aubergines and capsicum.

In certain embodiments, a saponin-containing plant extract may reduce methane production by altering rumen pH and/or reducing protozal methanogen symbionts.

In one embodiment, the composition can further comprise water. For example, the microorganism and/or growth by-products can be mixed with water and administered to the livestock animal. In another embodiment, the composition can be mixed with a livestock animal's drinking water as, for example, a feed additive and/or supplement. The drinking water composition can comprise, for example, 1 g/L to about 50 g/L of the microbe-based composition, about 2 g/L to about 20 g/L, or about 5 g/L to about 10 g/L.

Advantageously, the composition can enhance hydration and reduce the occurrence and/or severity of heat stress in livestock animals.

The composition can be formulated for enteral and/or parenteral delivery to the livestock animal's digestive system. For example, the composition can be formulated for oral administration via feed, water, and/or endoscopy; and/or for administration via direct injection into one or more parts of the digestive system (e.g., the rumen, stomach and/or intestines), via endoscopy, via enema, via fecal transplant, and/or via suppository.

In certain embodiments, the composition can further comprise one or more carriers and/or excipients suitable for delivery of the composition to the digestive system of the livestock animal, preferably, to the rumen, and can be formulated into preparations in, for example, solid, semi-solid, liquid or gaseous forms, such as tablets, capsules, pressed pellets, powders, granules, ointments, gels, lotions, solutions, suppositories, drops, patches, injections, inhalants and aerosols.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatisers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of carriers and/or excipients in the field of drugs and supplements is well known. Except for any conventional media or agent that is incompatible with the components of the subject compositions, its use in the subject compositions may be contemplated.

In one exemplary embodiment, the microbe-based composition can be formulated for direct administration into the digestive system or a part thereof via, for example, injection and/or endoscopy, for example, as a solution or suspension. The solution or suspension can comprise suitable non-toxic, enterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Water or saline solutions and aqueous dextrose and glycerol solutions may be preferably employed as carriers, particularly for enterally-injectable solutions.

In one exemplary embodiment, the microbe-based composition can be formulated for oral administration as a pre-made wet or dry feed, wherein the pre-made food has been cooked and/or processed to be ready for animal consumption. For example, the microorganism and/or growth by-products can be poured onto and/or mixed with the pre-made food, or the microorganism and/or growth by-products can serve as a coating on the outside of dry animal food pieces, e.g., morsels, kibbles or pellets.

In one embodiment, the composition can further comprise raw ingredients for making animal feed, wherein the raw ingredients, together with the microorganism and/or growth by-products, are then cooked and/or processed to make an enhanced dry or wet feed product. Raw ingredients Can include, for example, grains, grasses, roughage, forage, hay, straw, seeds, nuts, crop residue, vegetables, fruits, dried plant matter, and other flavorings, additives and/or sources of nutrients. In one embodiment, the composition is added to the raw food ingredients at a concentration of about 0.1% to about 50%, about 1% to about 25%, or about 5% to about 15% by weight.

The microbe-based composition can be added to the wet or day feed and/or raw feed ingredients at a concentration of, for example, about 0.1% to 99%, about 1% to about 75%, or about 5% to about 50% by weight.

As used herein, “dry food” refers to food that contains a limited moisture content, typically in the range of about 5% to about 15% or 20% w/v. Typically, dry processed food comes in the form of small to medium sized individual pieces, e.g., morsels, kibbles, treats, biscuits, nuts, cakes or pellets.

The supplemented dry food pieces can comprise consistent concentrations of the microbe-based composition per piece. In another embodiment, the composition can be utilized as a surface coating on the dry food pieces. Methods known in the art for producing dry processed foods can be used, including pressurized milling, extrusion, and/or pelleting.

In an exemplary embodiment, dry food may be prepared by, e.g., screw extrusion, which includes cooking, shaping and cutting raw ingredients into a specific shape and size in a very short period of time. The ingredients may be mixed into homogenous expandable dough and cooked in an extruder, and forced through a die under pressure and high heat. After cooking, the pellets are then allowed to cool, before optionally being sprayed with a coating. This coating may comprise, for example, liquid fat or digest, including liquid or powdered hydrolyzed forms of an animal tissue such as liver or intestine from, e.g., chicken or rabbit, and/or a nutritional oil. In other embodiments, the pellet is coated using a vacuum enrobing technique, wherein the pellet is subjected to vacuum and then exposed to coating materials after which the release of the vacuum drives the coating materials inside the pellet. Hot air drying can then be employed to reduce the total moisture content to 10% or less.

In one embodiment, the dry food is produced using a “cold” pelleting process, or a process that does not use high heat or steam. The process can use, for example, liquid binders with viscous and cohesive properties to hold the ingredients together without risk of denaturing or degrading important components and/or nutrients in the compositions of the subject invention.

In one embodiment, the composition can be applied to animal fodder, or cut and dried plant matter, such as hay, straw, silage, sprouted grains, legumes and/or grains.

In one embodiment, the composition may be prepared as a spray-dried biomass product. The biomass may be separated by known methods, such as centrifugation, filtration, separation, decanting, a combination of separation and decanting, ultrafiltration or microfiltration.

In one embodiment, the composition has a high nutritional content, for example, comprising up to 50% protein, as well as polysaccharides, vitamins, and minerals. As a result, the composition may be used as part of all of a complete animal feed composition. in one embodiment, the feed composition comprises the subject composition ranging from 15% of the feed to 99% of the feed.

In one embodiment, the subject composition can comprise additional nutrients to supplement an animal's diet and/or promote health and/or well-being in the animal, such as, for example, sources of amino acids (including essential amino acids), peptides, proteins, vitamins, microelements, fats, fatty acids, lipids, carbohydrates, sterols, enzymes, prebiotics, and minerals. In some embodiments, the microorganisms of the composition produce and/or provide these substances.

Preferred compositions comprise vitamins and/or minerals in any combination. Vitamins for use in a composition of this invention can include for example, vitamins A, E, K3, D3, B1, B3, B6, B12, C, biotin, folic acid, panthothenic acid, nicotinic acid, choline chloride, inositol and para-amino-benzoic acid. Minerals can include, for example, such as calcium, magnesium, phosphorus, potassium, sodium, chlorine, sulfur, chromium, cobalt, copper, iodine, iron, manganese, molybdenum, nickel, selenium, and zinc. Other components may include, but are not limited to, antioxidants, beta-glucans, bile salt, cholesterol, enzymes, carotenoids, and many others. Typical vitamins and minerals are those, for example, recommended for daily consumption and in the recommended daily amount (RDA), although precise amounts can vary. The composition would preferably include a complex of the RDA vitamins, minerals and trace minerals as well as those nutrients that have no established RDA, but have a beneficial role in healthy mammal physiology.

### Production of Microorganisms and/or Microbial Growth by-Products

The subject invention utilizes methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites, residual nutrients and/or intracellular components.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In one embodiment, one or more biostimulants may also be included, meaning substances that enhance the rate of growth of a microorganism. Biostimulants may be species-specific or may enhance the rate of growth of a variety of species.

In some embodiments, the method for cultivation may further comprise adding an antimicrobial in the medium before, and/or during the cultivation process.

In certain embodiments, an antibiotic can be added to a culture at low concentrations to produce microbes that are resistant to the antibiotic. The microbes that survive exposure to the antibiotic are selected and iteratively re-cultivated in the presence of progressively higher concentrations of the antibiotic to obtain a culture that is resistant to the antibiotic. This can be performed in a laboratory setting or industrial scale using methods known in the microbiological arts.

In certain embodiments, the amount of antibiotic in the culture begins at, for example, 0.0001 ppm and increases by about 0.001 to 0.1 ppm each iteration until the concentration in the culture is equal to, or about equal to, the dosage that would typically be applied to a livestock animal.

In certain embodiments, the antibiotics are those often used in livestock feed to promote growth and to help treat and prevent illness and infection in animals, such as, for example, procaine, penicillin, tetracyclines (e.g., chlortetracycline, oxytetracycline), tylosin, bacitracin, neomycin sulfate, streptomycin, erythromycin, monensin, roxarsone, salinomycin, tylosin, lincomycin, carbadox, laidlomycin, lasalocid, oleandomycin, virginamycin, and bambermycins. By producing beneficial microbes that are resistant to a particular livestock antibiotic, the microbes can be selected based on which antibiotic may be administered to the animal to treat or prevent a condition. Alternatively, an antibiotic can be selected for a livestock animal based on which beneficial microbe is being administered to the animal according to the subject methods so as not to harm the beneficial microbe.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The biomass content of the fermentation medium may be, for example, from 5 g/l to 180 g/l or more, or from 10 g/l to 150 g/l. The cell concentration may be, for example, at least 1×10⁹, 1×10¹⁰, 1×10¹¹, 1×10¹² or 1×10¹³ cells per gram of final product.

The microbial growth by-product produced by microorganisms of interest may be retained in the microorganisms or secreted into the growth medium. The medium may contain compounds that stabilize the activity of microbial growth by-product.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

### Preparation of Microbe-Based Products

A “microbe-based product,” is a product to be applied in practice to achieve a desired result. The microbe-based product can be simply a microbe-based composition harvested from a microbe cultivation process. Alternatively, a microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing a microorganism and/or the microbial metabolites produced by the microorganism and/or any residual nutrients. The product of fermentation may be used directly without extraction or purification. If desired, extraction and purification can be easily achieved using standard extraction and/or purification methods or techniques described in the literature.

The microorganisms in the microbe-based product may be in an active or inactive form. Furthermore, the microorganisms may be removed from the composition, and the residual culture utilized. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or solid substrate) resulting from the microbial growth can be removed from the growth vessel and transferred via, for example, piping for immediate use.

In one embodiment, the microbe-based product is simply the growth by-products of the microorganism. For example, biosurfactants produced by a microorganism can be collected from a submerged fermentation vessel in crude form, comprising, for example about 50% pure biosurfactant in liquid broth.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, solvents, biocides, other microbes and other ingredients specific for an intended use.

Other suitable additives, which may be contained in the formulations according to the invention, include substances that are customarily used for such preparations. Examples of such additives include surfactants, emulsifying agents, lubricants, buffering agents, solubility controlling agents, pH adjusting agents, preservatives, stabilizers and ultra-violet light resistant agents.

In one embodiment, the product may further comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, additional components such as an aqueous preparation of a salt, such as sodium bicarbonate or carbonate, sodium sulfate, sodium phosphate, or sodium biphosphate, can be included in the formulation.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used (e.g., a free-range cattle pasture). For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms can be generated, thereby requiring a smaller volume of the microbe-based product for use in the on-site application or which allows much higher density microbial applications where necessary to achieve the desired efficacy. This allows for a scaled-down bioreactor (e.g., smaller fermentation vessel, smaller supplies of starter material, nutrients and pH control agents), which makes the system efficient and can eliminate the need to stabilize cells or separate them from their culture medium. Local generation of the microbe-based product also facilitates the inclusion of the growth medium in the product. The medium can contain agents produced during the fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used (e.g., a livestock production facility), preferably within 300 miles, more preferably within 200 miles, even more preferably within 100 miles. Advantageously, this allows for the compositions to be tailored for use at a specified location. The formula and potency of microbe-based compositions can be customized for specific local conditions at the time of application, such as, for example, which animal species is being treated; what season, climate and/or time of year it is when a composition is being applied; and what mode and/or rate of application is being utilized.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products to improve GHG management.

The cultivation time for the individual vessels may be, for example, from 1 to 7 days or longer. The cultivation product can be harvested in any of a number of different ways.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### Methods for Reducing Greenhouse Gas Emissions

In preferred embodiments, the subject invention provides a method for reducing deleterious atmospheric gas emissions by reducing methane, carbon dioxide and/or other deleterious atmospheric gases, and/or precursors thereof (e.g., nitrogen and/or ammonia, which are precursors of nitrous oxide), produced in the digestive system and/or waste products of livestock animals.

“Livestock” animals, as used herein, are “domesticated” animals, meaning species that have been influenced, bred, tamed, and/or controlled over a sustained number of generations by humans, such that a mutualistic relationship exists between the animal and the human. Particularly, livestock animals include animals raised in an agricultural or industrial setting to produce commodities such as food, fiber and labor. Types of animals included in the term livestock can include, but are not limited to, alpacas, llamas, pigs (swine), horses, mules, asses, camels, dogs, ruminants, chickens, turkeys, ducks, geese, guinea fowl, and squabs.

In certain embodiments, the livestock animals are “ruminants,” or mammals that utilize a compartmentalized stomach suited for fermenting plant-based foods prior to digestion with the help of a specialized gut microbiome. Ruminants include, for example, bovines, sheep, goats, ibex, giraffes, deer, elk, moose, caribou, reindeer, antelope, gazelle, impala, wildebeest, and some kangaroos.

In specific exemplary embodiments, the livestock animals are bovine animals, which are ruminant animals belonging to the subfamily Bovinae, of the family Bovidae. Bovine animals can include domesticated and/or wild species. Specific examples include, but are not limited to, water buffalo, anoa, tamaraw, auroch, banteng, guar, gayal, yak, kouprey, domestic meat and dairy cattle (e.g., Bos taurus, Bos indicus), ox, bullock, zebu, saola, bison, buffalo, wisent, bongo, kudu, kewwel, imbabala, kudu, nyala, sitatunga, and eland.

In certain specific embodiments, the methods comprise contacting a microbe-based composition according to the subject invention with the digestive system of a livestock animal. The composition can be administered enterally and/or parenterally to, for example, the livestock animal's digestive system. For example, the composition can be administered to the livestock animal orally, via the livestock animal's feed, pasture and/or drinking water; via endoscopy; via direct injection into, e.g., the rumen, stomach, and/or intestines; via suppository; via fecal transplant; and/or via enema.

In certain embodiments, the composition can also be applied directly to the waste to reduce GHG emissions.

Advantageously, in preferred embodiments, the methods result in a reduction of methanogenic bacteria and/or protozoa present in the livestock animal's digestive system and/or waste. In certain embodiments, the methods can also result in a reduction of methane, carbon dioxide, other deleterious atmospheric gases, and/or precursors thereof, such as nitrogen and/or ammonia (precursors of nitrous oxide), in the livestock animal's digestive system and/or waste.

As used herein, “reduction” refers to a negative alteration of at least 0.25%, 0.5%, 1%, 5%, 10%, 15%, 20%, 25%, 30%, 35%, 40%, 45%, 50%, 55%, 60%, 70%, 75%, 80%, 85%, 90%, 95%, or 100%.

In some embodiments, the desired reduction is achieved within a relatively short time period, for example, within 1 week, 2 weeks, 3 weeks or 4 weeks of the animals ingesting the composition. In some embodiments, the desired reduction is achieved within, for example, 1 month, 2 months, 3 months, 4 months, 5 months or 6 months after employing the subject methods. In some embodiments, the desired reduction is achieved within 1 year, 2 years, 3 years, 4 years, or 5 years after employing the subject methods.

In some embodiments, the methods can further comprise adding materials to enhance the growth of the microorganisms of the subject composition at the time of application (e.g., adding nutrients and/prebiotics). In one embodiment, the nutrient sources can include, for example, sources of magnesium, phosphate, nitrogen, potassium, selenium, calcium, sulfur, iron, copper, zinc, proteins, vitamins and/or carbon. In certain embodiments, the livestock animal can be fed a source of prebiotics, which can include, for example, dry animal fodder, straw, hay, alfalfa, grains, forage, grass, fruits, vegetables, oats, and/or crop residue.

In some embodiments, prior to applying the composition, the method comprises assessing a livestock animal, herd of livestock animals, or livestock waste storage site for local conditions, determining a preferred formulation for the composition (e.g., the type, combination and/or ratios of microorganisms and/or growth by-products) that is customized for the local conditions, and producing the composition with said preferred formulation.

The local conditions can include, for example, age, health, size and species of the animal(s); herd size; purpose for producing the animal (e.g., meat, fur, fiber, labor, milk, etc.); species within the microbial population of an animal's gut and/or waste; environmental conditions, such as amount and type of GHG emissions, current climate, and/or season/time of year; mode and/or rate of application of the composition, and others as are deemed relevant.

After assessment, a preferred formulation for the composition can be determined so that the composition can be customized for these local conditions. The composition is then cultivated, preferably at a microbe growth facility that is within 300 miles, preferably within 200 miles, even more preferably within 100 miles of the location of application (e.g., an animal or livestock production facility, or a lagoon).

In some embodiments the local conditions are assessed periodically, for example, once annually, biannually, or even monthly. In this way, the composition formula can be modified in real time as necessary to meet the needs of the changing local conditions.

In an exemplary embodiment, the daily dosage of the composition administered to each animal is about 5 mg to about 100 grams, or about 10 mg to about 50 grams, or about 15 mg to about 25 grams, or about 20 mg to about 20 grams, or about 25 mg to about 10 grams, or about 30 mg to about 5 grams, per 100 kg of animal body weight.

In certain embodiments, the methods comprise adding the composition to drinking water and/or feed as a dietary supplement, The dietary supplement can have any suitable form such as a gravy, drinking water, beverage, yogurt, powder, granule, paste, suspension, chew, morsel, liquid solution, treat, snack, pellet, pill, capsule, tablet, sachet, or any other suitable delivery form. The dietary supplement can comprise the subject microbe-based compositions, as well as optional compounds such as vitamins, minerals, probiotics, prebiotics, and antioxidants. In some embodiments, the dietary supplement may be admixed with a feed composition or with water or other diluent prior to administration to the animal.

In some embodiments, the composition is applied to a grazing field or pasture as well as to the drinking water and/or feed.

According to the methods of the subject invention, administration of the microbe-based compositions can be performed as part of a dietary regimen, which can span a period ranging from parturition through the adult life of the animal. In certain embodiments, the animal is a young or growing animal. In some embodiments, the animal is an aging animal. In other embodiments administration begins, for example, on a regular or extended regular basis, when the animal has reached more than about 30%, 40%, 50%, 60%, or 80% of its projected or anticipated lifespan.

In some embodiments, the methods of the subject invention can be utilized by a livestock producer or waste processor for reducing carbon credit usage. Thus, in certain embodiments, the subject methods can further comprise conducting measurements to assess the effect of the method on reducing the generation of carbon dioxide and/or other deleterious atmospheric gases, and/or precursors thereof (e.g., nitrogen and/or ammonia), and/or to assess the effect of the method on the control of methanogens in the livestock animal's digestive system and/or waste, using standard techniques in the art.

These measurements can be conducted according to known methods in the art (see, e.g., Storm et al. 2012, incorporated herein by reference), including, for example, gas capture and quantification, chromatography, respiration chambers (which measure the amount of methane exhaled by an individual animal), and in vitro gas production technique (where feed is fermented under controlled laboratory and microbial conditions to determine amount of methane and/or nitrous oxide is emitted per gram of dry matter). The measurements can also come in the form of testing the microbial population in an animal, for example, by sampling milk, feces, and/or stomach contents and using, for example, DNA sequencing and/or cell plating to determine the number of methanogenic microbes present therein.

Measurements can be conducted at a certain time point after application of the microbe-based composition. In some embodiments, the measurements are conducted after about 1 week or less, 2 weeks or less, 3 weeks or less, 4 weeks or less, 30 days or less, 60 days or less, 90 days or less, 120 days or less, 180 days or less, and/or 1 year or less.

Furthermore, the measurements can be repeated over time. In some embodiments, the measurements are repeated daily, weekly, monthly, bi-monthly, semi-monthly, semi-annually, and/or annually.

### Treating Livestock Waste

In certain specific embodiments, a composition according to embodiments of the subject invention is administered directly to a manure lagoon, waste pond, tailing pond, tank or other storage facility where livestock and/or food processing waste is stored and/or treated.

Advantageously, in some embodiments, the microorganisms in the composition, e.g., B. amy, can facilitate increased decomposition of manure while reducing the amount of GHG emitted therefrom, e.g., methane, carbon dioxide and/or nitrous oxide. Furthermore, in some embodiments, applying the composition to manure enhances the value of the manure as an organic fertilizer due to the ability of the microorganisms to inoculate the soil of a field or crop to which the manure is eventually applied. The microorganisms and their growth by-products can improve soil biodiversity, enhance rhizosphere properties, and enhance plant growth and health, which can lead to, for example, a reduced need for nitrogen-rich synthetic fertilizers.

In some embodiments, the lagoon or waste pond comprises other animal and/or food processing waste by-products, for example, palm oil processing waste (e.g., palm oil mill effluent), olive oil processing waste (e.g., olive press cake and olive mill wastewater), dairy processing waste (e.g., acid-whey), and slaughterhouse waste (e.g., livestock carcass remnants). These high-fat waste products produce pollution and foul odors, and can form semi-solid fat layers on top of wastewater, which encourages the growth of GHG-producing microorganisms.

In certain embodiments, the microorganisms of the subject compositions can help metabolize the fat layer and increase the decomposition rate, in addition to providing GHG-reducing benefits as described previously.

In certain embodiments, the method comprises supplementing the composition with a biosurfactant, e.g., a rhamnolipid and/or a sophorolipid, which can enhance the breakdown of fats and enhance the control of GHG-producing microorganisms.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1—In Vitro Testing

Compositions according to embodiments of the subject invention were screened for their ability to reduce enteric methane and carbon dioxide emissions in cattle. Twenty-four vessels were filled with cattle rumen fluid, artificial saliva, 1 g rumen solids, 1 g super basic ration and 1% by volume of a treatment composition. Triplicates of eight treatments were performed, including one control triplicate.

### Treatments Included:

0—Control
1—B. amy
2—P. ostreatus
3—S. boulardii
4—B. amy+P. ostreatus
5—B. amy+S. boulardii
6—P. ostreatus+S. boulardii
7—B. amy+P. ostreatus+S. boulardii

After 24 hours, the amount of methane, carbon dioxide and total gas volumes (ml/gDM) collected from each vessel was measured.

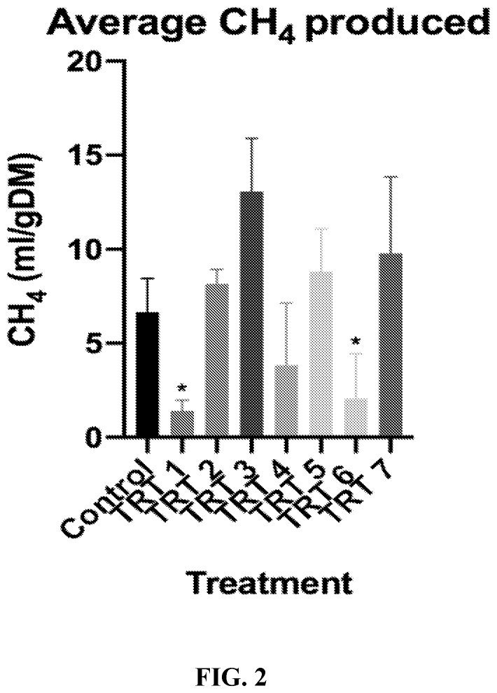

FIG. 2 shows the results for methane. Treatment 1, comprising B. amy, showed a 78% reduction (p=0.05) in average amount of methane gas compared to the control. Treatment 6, comprising S. boulardii and P. ostreatus, showed a 69% reduction (p=0.03) in average amount of methane gas compared to the control.

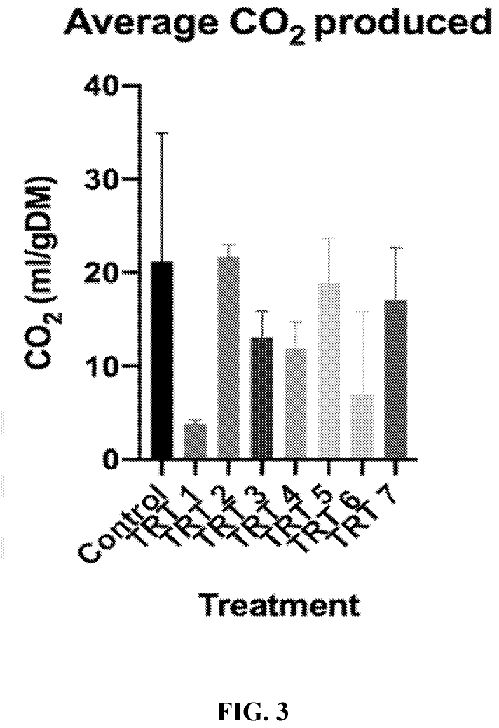

FIG. 3 shows the results for carbon dioxide reduction. Treatment 1, comprising B. amy, showed the greatest reduction in average amount of carbon dioxide gas compared to the control, and Treatment 6, comprising S. boulardii and P. ostreatus, showed the next greatest reduction.

### Example 2—Additional In Vitro Testing

Treatment #1 from Example 1 above comprising B. amy was screened at variable inclusion rates for its ability to reduce enteric methane and carbon dioxide emissions in cattle. Eight replicates each of five different inclusion rates were conducted in individual vessels (equaling 40 vessels total). The vessels comprised rumen fluid, artificial saliva, 1 g rumen solids, 1 g super basic ration, and a variable inclusion rate of Treatment #1. The variable inclusion rates were: 0% (control), 0.1%, 0.2%, 0.5% and 1%.

Twenty-four hours after initiation of in-vitro rumen fermentation, the amount of methane, carbon dioxide and total gas volumes (ml/gDM) collected from each vessel was measured.

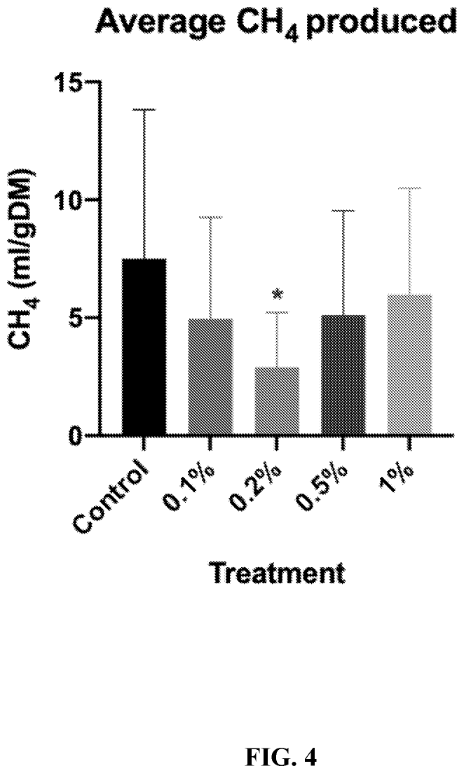

FIG. 4 shows the results for methane. The treatment comprising an inclusion rate of 0.2% B. amy, showed the greatest reduction in CH₄ emissions. (* indicates a significant reduction, p=0.0174).

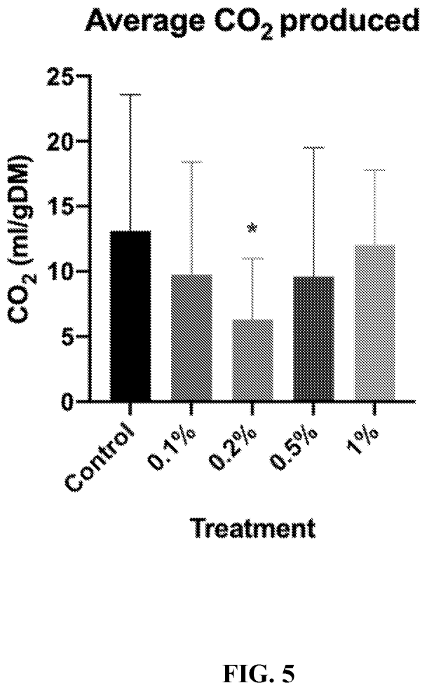

FIG. 5 shows the results for carbon dioxide. The treatment comprising an inclusion rate of 0.2% B. amy, showed the greatest reduction in CO₂ emissions. (* indicates a significant reduction, p=0.0491).

### Example 3—B. amy Product

One microbe-based product of the subject invention comprises B. amy. B. amy inoculum is grown in a small-scale reactor for 24 to 48 hours. Myxococcus xanthus inoculum is grown in a 2 L working volume seed culture flask for 48 to 120 hours. A fermentation reactor is inoculated with the two inocula. Nutrient medium is fed to the fermentation reactor continuously from a feed tank. The nutrient medium comprises:

| | |
|---|---|
| Glucose | 1 g/L to 5 g/L |
| Casein peptone | 1 g/L to 10 g/L |
| K₂HPO₄ | 0.01 g/L to 1.0 g/L |
| KH₂PO₄ | 0.01 g/L to 1.0 g/L |
| MgSO₄•7H₂O | 0.01 g/L to 1.0 g/L |
| NaCl | 0.01 g/L to 1.0 g/L |
| CaCO₃ | 0.5 g/L to 5 g/L |
| Ca(NO₃)₂ | 0.01 g/L to 1.0 g/L |
| Yeast extract | 0.01 g/L to 5 g/L |
| MnCl₂•4H₂O | 0.001 g/L to 0.5 g/L |
| Teknova trace element | 0.5 ml/L to 5 ml/L |

Fine grain particulate anchoring carrier is suspended in the nutrient medium. The carrier comprises cellulose (1.0 to 5.0 g/L) and/or corn flour (1.0 to 8.0 g/L).

pH in the reactor is maintained at about 6.8; temperature is maintained at about 24° C.; DO is maintained at about 50%; and air flow rate is maintained at about 1 vvm.

A foam layer comprising microbial growth by-products is produced during fermentation and is purged out and collected in a container comprising a pH meter. The pH meter is used to monitor the pH of the foam: if the pH varies outside of the range of 2.0 to 3.0, pH adjusters are added to bring the pH back within that range for long-term preservation of the metabolites therein. Foam continues to be produced, purged from the reactor, and collected for 7 days or longer (e.g., indefinitely).

Sampling of the fermenter and the foam collection tank for CFU count, sporulation percentage and/or purity is performed at 0 hr., then twice per day throughout fermentation. Sampling can also occur at the time that foam is purged and collected. When/if sporulation percentage of the bacterial culture is detected (using microscope slide estimation) to be greater than 20%, additional nutrient media is added to the fermenter. LC-MS analysis is carried out on acidified lipopeptide samples from the foam collection tank. The samples are stored at about 4° C.

The fermentation cycle is continued for at least one week, with nutrient medium feeding and foam collection occurring until, for example, foam can no longer be extracted from the fermenter. Lipopeptide production is observed in as little as 3 hours after inoculation, with a total yield reaching 20 to 30 g/L per week (or 250 dry kg of lipopeptide per week). The yield from this method can reach up to 10 times greater than traditional, non-antagonistic methods of cultivation B. amyloliquefaciens.

### Concentration and Drying of Product

The cell biomass, comprising B. amy spores, is collected and dried to a residual moisture no higher than 8%. The remaining cell-free foam and/or supernatant, which can reduce surface tension to 29-30 mN/m at 200 ppm, is evaporated using industrial evaporators to obtain a highly-viscous liquid containing biosurfactants and other metabolites. The viscous compound is then dried to produce a powder, which is milled and mixed with the dry spores at a ratio of 1 g to 50 mg, spores to supernatant.

The final product preferably contains no less than 100 billion spores per gram. The ideal treatment for cattle is 1 g of the composition per head of cattle per day, or if applied to a pasture, 1 g per 100 sq. feet of pasture per week.

### Example 4—P. ostreatus and S. boulardii Product

One microbe-based product of the subject invention comprises P. ostreatus and S. boulardii.

P. ostreatus can be produced using large scale submerged fermentation vessels having a volume of 500 L to 2800 L. The fermentation cycle is about 10 days, with an average biomass production yield of about 1.7×10⁶ cells/g. As an example, these yields produce a lovastatin content of >13% by weight.

S. boulardii can be produced using large scale submerged fermentation vessels having a volume of 500 L to 2800 L. The fermentation cycle is about 16 hours, with an average biomass production yield of about 2.3×10⁹ cells/g.

The two microbe-based products can be mixed and dried to produce a composition such that 1 daily dose for one head of cattle comprises 150 mg of P. ostreatus and 10 g of S. boulardii.

### Example 5—D. hansenii Product

One microbe-based product of the subject invention comprises D. hansenii. One dosage of the product comprises 5×10¹⁰ CFU per day for one head of cattle.

### REFERENCES

Government of Western Australia. (2018). “Carbon farming: reducing methane emissions from cattle using feed additives.” https://www.agric.wa.gov.au/climate-change/carbon-farming-reducing-methane-emissions-cattle-using-feed-additives. (“Carbon Farming 2018”).
Gerber, P. J., et al. (2013). “Tackling climate change through livestock—A global assessment of emissions and mitigation opportunities.” Food and Agriculture Organization of the United Nations, Rome. Viewed Apr. 5, 2019. http://www.fao.org/3/i3437e/i3437e.pdf. (“Gerber et al. 2013”).
Holtshausen, L. et al. (2009). “Feeding saponin-containing Yucca schidigera and Quillaja saponaria to decrease enteric methane production in dairy cows.” J. Dairy Sci. 92:2809-2821.
Ishler, V. A., (2016). “Carbon, Methane Emissions and the Diary Cow.” Penn State College of Agricultural Sciences. https://extension.psu.edu/carbon-methane-emissions-and-the-dairy-cow. (“Ishler 2016”).
Patra, A., Park, T., Kim, M. et al. (2017). Rumen methanogens and mitigation of methane emission by anti-methanogenic compounds and substances. J Animal Sci Biotechnol 8, 13. https://doi.org/10.1186/s40104-017-0145-9 (“Patra et al. 2017”).
Pidwirny, M. (2006). “The Carbon Cycle”. Fundamentals of Physical Geography, 2nd Edition. Viewed Oct. 1, 2018. http://www.physicalgeography.net/fundamentals/9r.html. (“Pidwirny 2006”).
Storm, Ida M. L. D., A. L. F. Hellwing, N. I. Nielsen, and J. Madsen. (2012). “Methods for Measuring and Estimating Methane Emission from Ruminants.” Animals (Basel). June 2(2): 160-183. doi: 10.3390/ani2020160.
United States Environmental Protection Agency. (2016). “Climate Change Indicators in the United States.” https://www.epa.gov/sites/production/files/2016-08/documents/climate_indicators_2016.pdf. (“EPA Report 2016”).
United States Environmental Protection Agency. (2016). “Overview of Greenhouse Gases.” Greenhouse Gas Emissions. https://www.epa.gov/ghgemissions/overview-greenhouse-gases. (“Greenhouse Gas Emissions 2016”).

1. A method for reducing a deleterious atmospheric gas and/or a precursor thereof produced in a livestock animal's digestive system and/or waste, the method comprising contacting a composition comprising one or more beneficial microorganisms and/or one or more microbial growth by-products with the livestock animal's digestive system,
wherein the one or more beneficial microorganisms are Bacillus amyloliquefaciens, Pleurotus ostreatus, Saccharomyces boulardii, Debaryomyces hansenii, Lentinula edodes, Trichoderma viridae, Wickerhamomyces anomalus, Saccharomyces cerevisiae, Starmerella bombicola, Meyerozyma guilliermondii, Pichia occidentalis, Monascus purpureus, Acremonium chrysogenum, Myxococcus xanthus, Bacillus subtilis, Bacillus subtilis strain “B4” and/or Bacillus licheniformis.

2. (canceled)

3. The method of claim 1, wherein the composition is administered directly to the digestive system orally, via endoscopy, via fecal transplant, via suppository, via enema or via injection into the stomach, rumen and/or intestines.

4. (canceled)

5. The method of claim 1, wherein the deleterious atmospheric gas is methane and/or carbon dioxide, and/or wherein the deleterious atmospheric gas precursor is nitrogen and/or ammonia.

6. (canceled)

7. The method of claim 1, wherein a methanogenic bacterium and/or a protozoan in the livestock animal's digestive system is controlled.

8. (canceled)

9. The method of claim 1, further comprising administering a saturated long chain fatty acid with the one or more beneficial microorganisms and/or one or more microbial growth by-products, wherein the saturated long chain fatty acid is stearic acid, palmitic acid and/or myristic acid.

10. (canceled)

11. The method of claim 1, further comprising administering one or more of the following components with the one or more beneficial microorganisms and/or one or more microbial growth by-products: seaweed (Asparagopsis taxiformis); kelp; 3-nitrooxypropanol; anthraquinones; ionophores selected from monensin and lasalocid; polyphenols selected from saponins and tannins; Yucca schidigera extract; Quillaja saponaria extract; organosulfurs; garlic extract; flavonoids selected from quercetin, rutin, kaemferol, naringin, and anthocyanidins; bioflavonoids isolated from green citrus fruits, rose hips and/or black currants; carboxylic acid; and terpenes selected from d-limonene, pinene and citrus extracts.

12. The method of claim 1, wherein the beneficial microorganism is Bacillus amyloliquefaciens NRRL B-67928 (“B. amy”) and/or Bacillus subtilis NRRL B-68031 (“B4”).

13. (canceled)

14. The method of claim 1, wherein the beneficial microorganism is Pleurotus ostreatus.

15-17. (canceled)

18. The method of claim 1, wherein the one or more beneficial microorganisms are B. amy, S. boulardii, P. ostreatus and D. hansenii.

19. The method of claim 1, wherein the beneficial microorganism is W. anomalus.

20-22. (canceled)

23. The method of claim 1, wherein the one or more microbial growth by-products are in crude form, said crude form comprising a supernatant resulting from fermentation of a microorganism that produces said growth by-products.

24. The method of claim 1, wherein the one or more microorganisms and/or one or more microbial growth by-products are applied to drinking water and/or to feed that the livestock animal ingests.

25. The method of claim 1, further comprising assessing the effect of the method on the reduction of enteric deleterious atmospheric gas emissions and/or precursors thereof in the livestock animal's digestive system and/or waste.

26. The method of claim 1, further comprising assessing the effect of the method on the control of methanogenic bacteria and/or protozoa in the livestock animal's digestive system and/or waste.

27. The method of claim 1, used for reducing the number of carbon credits used by an operator involved in livestock production.

28. A composition for reducing enteric deleterious atmospheric gases and/or precursors thereof in a livestock animal's digestives system, the composition comprising one or more beneficial microorganisms and/or one or more microbial growth by-products,
wherein the one or more beneficial microorganisms are Bacillus amyloliquefaciens, Pleurotus ostreatus, Saccharomyces boulardii, Debaryomyces hansenii, Lentinula edodes, Trichoderma viridae, Wickerhamomyces anomalus, Saccharomyces cerevisiae, Starmerella bombicola, Meyerozyma guilliermondii, Pichia occidentalis, Monascus purpureus, Acremonium chrysogenum, Myxococcus xanthus, Bacillus subtilis, Bacillus subtilis strain “B4,” and/or Bacillus licheniformis.

29. The composition of claim 28, wherein the beneficial microorganism is a strain of Bacillus amyloliquefaciens; wherein the strain of Bacillus amyloliquefaciens is B. amyloliquefaciens NRRL B-67928 (“B. amy”).

30-38. (canceled)

39. The composition of claim 28, further comprising a saturated long chain fatty acid selected from stearic acid, palmitic acid and myristic acid.

40. The composition of claim 28, further comprising a germination enhancer selected from L-alanine, L-leucine and manganese.

41. The composition of claim 28, further comprising one or more of the following components: seaweed (Asparagopsis taxiformis and/or Asparagopsis armata); kelp; nitrooxypropanols; anthraquinones; ionophores selected from monensin and lasalocid; polyphenols selected from saponins and tannins; Yucca schidigera extract; Quillaja saponaria extract; organosulfurs; garlic extract; flavonoids selected from quercetin, rutin, kaemferol, naringin, and anthocyanidins; bioflavonoids isolated from green citrus fruits, rose hips and/or black currants; carboxylic acid; and terpenes selected from d-limonene, pinene and citrus extracts.

42-44. (canceled)

45. A method for reducing greenhouse gas emissions from livestock waste products, the method comprising administering a composition according to claim 28 to the livestock animal's digestive system.

46. A method for reducing greenhouse gas emissions from livestock waste products, the method comprising administering a composition according to claim 28 to the waste products.

47. The method of claim 46, wherein the composition is applied to a lagoon, tailing pond or storage tank used for storage and/or treatment of livestock waste.

48. The method of claim 46, further comprising applying the waste products to a field or crop as an organic fertilizer.

49. The method of claim 48, used to reduce the amount of nitrogen-rich fertilizer that is applied to the field or crop.

50. A method for reducing greenhouse gas emissions from livestock waste products, the method comprising administering a sophorolipid biosurfactant to the waste products and, optionally, B. amy.
